(19) United States
(12) Patent Application Publication
(10) Pub. No.: US 2017/0056351 A1
(43) Pub. Date: Mar. 2, 2017

(54) COMPOSITION FOR INDUCING TUMOR IMMUNITY

(71) Applicants: UNIVERSITY OF OCCUPATIONAL AND ENVIRONMENTAL HEALTH, JAPAN, Fukuoka (JP); SBI PHARMACEUTICALS CO., LTD., Tokyo (JP)

(72) Inventors: Shigeru NISHIZAWA, Fukuoka (JP); Junkoh YAMAMOTO, Fukuoka (JP); Tohru TANAKA, Tokyo (JP)

(21) Appl. No.: 15/120,114

(22) PCT Filed: Feb. 19, 2015
(86) PCT No.: PCT/JP2015/054548

§ 371 Date: Aug. 18, 2016

(30) Foreign Application Priority Data
Feb. 25, 2014 (JP) 2014034669

(51) Int. Cl. A61K 31/197; A61N 5/10; A61K 45/06

(52) U.S. Cl. CPC A61K 31/197; A61K 45/06; A61N 5/10; A61N 2005/1098

(57) ABSTRACT

[Problem to be Solved]

It is an object of the present invention to provide a composition, which induces immunity to tumor cells and is also able to induce inhibition of tumor growth.

[Solution]

Provided is a composition for inducing tumor immunity, comprising ALAs.

### TECHNICAL FIELD

The present invention relates to a composition for inducing tumor immunity, etc., which comprises ALAs.

### BACKGROUND ART

Radiation therapy is a method for treating cancer by applying electron beam or particle beam to the cancer, and it is one of the major three cancer treatment methods, which is alongside with surgery and anti-cancer therapy. As electron beam used in the radiation therapy, X-rays or γ-rays have been practically used, and as particle beam, heavy ion beam, proton beam, and neutron beam have been practically used. In such radiation therapy, the larger the exposure dose, the higher the cancer-killing effects that can be naturally obtained. However, such a large exposure dose is problematic in terms of radiation injury to normal cells. The term “radiation injury” is a generic name for physical damages or injuries caused by being exposed to radiation. Irradiation, radioactive contamination and the like have been known as causes of radiation injury. The radiation injury includes: early radiation injury (acute radiation injury) that is radiation injury having acute symptoms appearing immediately after radiation exposure or within several months after the exposure; and late radiation injury (delayed radiation injury) that is radiation injury appearing several years to several tens of years after radiation exposure.

The sensitivity of cells to radiation becomes higher, when the cells are actively dividing, and the cell regeneration system, such as a hematopoietic organ, is most susceptible. For example, regarding early radiation injury, when people are exposed to a radiation of 1 Gy (gray) or more, some of them have symptoms named “radiation sickness,” which are similar to the symptoms of hangover, such as nausea, vomiting and general malaise. When people are exposed to a radiation of 1.5 Gy or more, hematopoietic cells having the highest sensitivity to radiation are susceptible, bleeding increasingly occurs due to suspension of the supply of leukocytes and platelets, and immunity is decreased. In the case of severe symptoms, a subject who has exposed to radiation dies approximately 30 to 60 days after the exposure. Moreover, in the case of skin, epithelial basal cells are highly susceptible. When the skin is exposed to a radiation of 3 Gy or more, symptoms such as hair removal and temporary erythema appear. In the case of exposure to a radiation of 7 to 8 Gy, blister formation is observed, and in the case of exposure to a radiation of 10 Gy or more, ulcer is observed. When a body is exposed to a radiation of 5 Gy or more, stem cells in the small intestine die, and the supply of absorptive cells is terminated. As a result, diarrhea caused by a reduction in absorption ability, or bacterial infection is developed, and in the case of severe symptoms, the subject who has exposed to radiation dies within 20 days after the exposure. In the case of exposure to a radiation at an extremely high dose such as 15 Gy or more, such high radiation exposure has effects on the central nervous system, and disturbance of consciousness and shock symptom can be seen. The influence of radiation exposure on the central nervous system appears promptly, and almost all of subjects who have exposed to radiation die within 5 days after the exposure. On the other hand, in the case of late radiation injury, the rate of incidence of various malignant tumors including leukemia as a typical example, radiation cataract, and the like is increased.

To date, the present inventors had proposed a technique of preventing or reducing radiation injury, and had contributed to utilization of radiation therapy (Patent Literature 1).

Moreover, as another approach, an attempt to enhance the cancer-killing effects of radiation therapy by utilizing an enhancer, even though the radiation is applied at a low dose, has been known. Radiation therapy requires oxygen to exhibit its effects. Since cancer (in a tumor) is generally hypoxic, a radiosensitizer, which is decomposed by irradiation to generate oxygen, has been often used (Non Patent Literature 1). Furthermore, studies for focusing on the fact that porphyrins such as hematoporphyrin are accumulated in larger amounts in cancer cells than in normal cells, and performing radiation therapy using such porphyrins, have also been known (Patent Literature 2).

On the other hand, 5-aminolevulinic acids (which are also referred to as “ALAs” in the present description), which are precursor substances of porphyrin and are biological substances, have been known as intermediates in a tetrapyrrole biosynthetic pathway, which are broadly present in animals, plants, and fungi. In general, 5-aminolevulinic acid is biosynthesized from succinyl CoA and glycine by the action of 5-aminolevulinic acid synthase. It has been known that ALAs themselves do not have photosensitivity, but the ALAs are metabolically activated in cells by a series of enzymes in a heme biosynthetic pathway so that they are converted to porphyrins (which are mainly protoporphyrin IX (PpIX)). In addition, such PpIX has been known as a photosensitizer having peaks at 410 nm, 510 nm, 545 nm, 580 nm, 630 nm, etc. Since ALA is metabolized in a living body and is then discharged from the body within 48 hours, the ALA is characterized in that it has almost no influences on the photosensitivity of the body as a whole. As such, photodynamic therapy or photodynamic treatment (which is also referred to as “ALA-PDT”), in which ALAs are used, has been developed, and these treatments have attracted attention as therapies that have low invasiveness and are capable of maintaining QOL. Tumor diagnostic and/or therapeutic agents and the like, in which ALAs are used, have been reported. Further, ALAs have also been known to be useful as agents for preventing, improving or treating adult disease, cancer, and male infertility (for example, Patent Literature 3 to 5).

Since PpIX is mainly accumulated in tumor cells upon administration of ALA to the tumor cells, previous studies had been conducted directed towards using the ALA as an enhancer of radiation therapy, so far. However, from such studies, inconsistent results had been obtained, and thus, the usefulness thereof had not been clarified (Non Patent Literature 2, Non Patent Literature 3, and Non Patent Literature 4). In Non Patent Literature 2, it has been, unfortunately, demonstrated that the ALA is not useful as an enhancer of radiation therapy. In Non Patent Literature 3, all of the experiments have been carried out in vitro, and different experimental results have been shown, depending on the types of cell lines. Thus, the usefulness of ALA as an enhancer of radiation therapy has not been necessarily clarified. Even in Non Patent Literature 4, experiments have been carried out in vitro, and only a little radiosensitization action has been obtained from one time of irradiation and administration of ALA. In Patent Literature 2, all of the experiments have been carried out in vitro, and also, experimental data regarding ALA have not been disclosed at all. In addition, X-ray has been applied only once.

Hence, from the prior art techniques, the usefulness of ALA as an enhancer of radiation therapy has not been known. Moreover, regarding the relationship between radiation and ALA, these technical publications have only explained cytotoxic effects on tumor cells based on in vitro reactive oxygen species or free radicals, even if the publications have disclosed it at maximum.

Further, in Non Patent Literature 5, ALA-PDT has been carried out on tumor (lung cancer tumor)-transplanted mice, the tumor has been then isolated, immunocompetent cells have been then allowed to directly come into contact with the tumor in vitro, and thereafter, infiltration of the cells in the tumor has been confirmed. However, Non Patent Literature 4 has just focused on the photodynamic action of PDT, and thus, this publication does not describe at all the effects of ALA alone (without light irradiation), the usefulness of ALA as an enhancer of radiation therapy, the presence or absence of inhibition of tumor growth, etc.

### CITATION LIST

### Patent Literature

[Patent Literature 1] WO2014/017046
[Patent Literature 2] Japanese Patent Laid-Open No. 2009-155239
[Patent Literature 3] WO2010/050179
[Patent Literature 4] Japanese Patent Laid-Open No. 2011-16753
[Patent Literature 5] WO2009/139156

### Non Patent Literature

[Non Patent Literature 1] Transl Oncol. 2011 August; 4(4):189-98. Epub 2011 Aug. 1. Six degrees of separation: the oxygen effect in the development of radiosensitizers. Oronsky BT1, Knox S J, Scicinski J.
[Non Patent Literature 2] Schaffer M, Schaffer P M, Jori G, Corti L, Sotti G, Hofstetter A, et al.: Radiation therapy combined with photofrin or 5-ALA: effect on Lewis sarcoma tumor lines implanted in mice. Preliminary results. Tumori 88: 407-10, 2002.
[Non Patent Literature 3] Yamamoto J, Ogura S, Tanaka T, Kitagawa T, Nakano Y, Saito T, et al.: Radiosensitizing effect of 5-aminolevulinic acid-induced protoporphyrin IX in glioma cells in vitro. Oncol Rep 27: 1748-52, 2012.
[Non Patent Literature 4] Berg K, et al. Combined treatment of ionizing radiation and photosensitization by 5-aminolevlulinic acid-induced protoporphyrin IX. Radiation Res. 1995 June; 142(3): 340-346
[Non Patent Literature 5] Skivka L M, Gorobets O B, Kutsenok V V, Lozinsky M O, Borisevich A N, Fedorchuk A G, et al.: 5-aminolevulinic acid mediated photodynamic therapy of Lewis lung carcinoma: a role of tumor infiltration with different cells of immune system. Exp Oncol 26: 312-5, 2004.

### SUMMARY OF INVENTION

### Technical Problem

It is an object of the present invention to provide a composition capable of inducing tumor immunity and also capable of inducing inhibition of tumor growth.

### Solution to Problem

The present inventors have precisely analyzed the studies for enhancing the effects of radiation therapy by combining ALA administration with the radiation therapy. In the process of such analysis, the inventors have surprisingly discovered from an in vivo experiment that even a single administration of ALA is able to induce or enhance the anti-tumor immune response of a host.

Moreover, the present inventors have focused on the characteristic of ALA as an intrinsic substance, which is relevant to immunity as a precursor of heme, and the inventors have conducted a combination of ALA administration and irradiation several times. As a result, the inventors have found that, in addition to direct cytotoxic effects provided by irradiation, even after the irradiation, ALA contributes to induction of long-term immunological effects, such as an increase in delayed reactive oxygen species production in the tumor and induction of tumor cytotoxic M1 macrophages, thereby efficiently inhibiting the growth of the tumor.

Furthermore, as a result of intensive studies, the present inventors have surprisingly found that, focusing on the action mechanism of antigen presentation, when a radiation that is combined with ALA administration is applied at low energy dividedly over several applications, the sensitizing effects of the ALA in radiation therapy are significantly enhanced, in compared to a case where the radiation is applied once at high energy, thereby completing the present invention.

(1) Specifically, in one embodiment, the present invention relates to a composition for inducing tumor immunity, comprising a compound represented by the following formula (I), or a salt thereof:

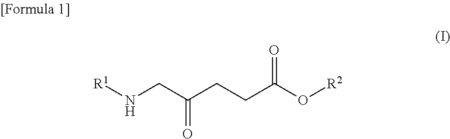

wherein R¹ represents a hydrogen atom or an acyl group, and R² represents a hydrogen atom, a linear or branched alkyl group, a cycloalkyl group, an aryl group, or an aralkyl group.
(2) In addition, in one embodiment, the present invention relates to the above described composition for inducing tumor immunity, which is used in an aspect that does not involve light irradiation to tumor.
(3) Moreover, in one embodiment, the present invention relates to the above described composition for inducing tumor immunity, which is administered to a mammal.
(4) Furthermore, in one embodiment, the present invention relates to the above described composition for inducing tumor immunity, which is repeatedly administered to the mammal at least two or more times.
(5) Further, in one embodiment, the present invention relates to the above described composition for inducing tumor immunity, wherein a tumor cytotoxic M1 macrophages is induced in a tumor cell in the mammal.
(6) Further, in one embodiment, the present invention relates to the above described composition for inducing tumor immunity, which is used to enhance therapeutic effect of radiation therapy.
(7) Still further, in one embodiment, the present invention relates to the above described composition for inducing tumor immunity, wherein, in the radiation therapy, radiation is repeatedly applied to the tumor cell in the mammal at least two or more times.
(8) Still further, in one embodiment, the present invention relates to the above described composition for inducing tumor immunity, which further comprises an iron compound.
(9) Otherwise, in one embodiment, the present invention relates to the above described composition for inducing tumor immunity, wherein the iron compound is administered to the mammal in combination.
(10) In addition, in one embodiment, the present invention relates to the above described composition for inducing tumor immunity, wherein the iron compound is one or two or more compounds selected from the group consisting of ferric chloride, iron sesquioxide, iron sulfate, ferrous pyrophosphate, ferrous citrate, iron sodium citrate, ferrous sodium citrate, iron ammonium citrate, ferric pyrophosphate, iron lactate, ferrous gluconate, sodium iron diethylenetriamine pentaacetate, ammonium iron diethylenetriamine pentaacetate, sodium iron ethylenediamine tetraacetate, ammonium iron ethylenediamine tetraacetate, sodium iron dicarboxymethylglutamate, ammonium iron dicarboxymethylglutamate, ferrous fumarate, iron acetate, iron oxalate, ferrous succinate, sodium iron succinate citrate, heme iron, dextran iron, triethylenetetramine iron, lactoferrin iron, transferrin iron, sodium iron chlorophyllin, ferritin iron, saccharated iron oxide, and ferrous glycine sulphate.
(11) Moreover, in one embodiment, the present invention relates to the above described composition for inducing tumor immunity, wherein the tumor is brain tumor.
(12) Furthermore, in another embodiment, the present invention relates to a medicament for preventing or treating cancer, comprising a combination of (i) the composition for inducing tumor immunity according to any one of the aforementioned (1) to (11), and (2) an anticancer agent, which are simultaneously or sequentially administered.
(13) Further, in one embodiment, the present invention relates to the above described medicament for preventing or treating cancer, wherein the combination aspect is a compounding agent or a kit.
(14) Further, in another embodiment, the present invention relates to a combination of (i) the composition for inducing tumor immunity according to any one of the aforementioned (1) to (11), and (ii) an anticancer agent, for use in preventing or treating cancer, wherein

(i) the composition for inducing tumor immunity and (ii) the anticancer agent are simultaneously or sequentially administered.

(15) Still further, in another embodiment, the present invention relates to a method for inducing tumor immunity in a subject affected with cancer, which comprises:

(A) a step of administering, once or several times, to a subject affected with cancer, the compound represented by the above described formula (I) or a salt thereof; and

(B) optionally, a step of applying radiation to the cancer in the subject once or several times.

(16) Still further, in one embodiment, the present invention relates to the above described method for inducing tumor immunity, which further comprises:

(C) a step of simultaneously or sequentially administering an anticancer agent to the subject.

(17) Still further, in another embodiment, the present invention relates to use of the compound represented by the above described formula (I) or a salt thereof, for producing a medicament for inducing tumor immunity.
(18) An invention made by arbitrarily combining the aforementioned one or multiple characteristics of the present invention is naturally included in the scope of the present invention, unless it includes a technical contradiction.

### Advantageous Effects of Invention

In one aspect, the composition for inducing tumor immunity of the present invention can provide a novel action mechanism, by which an increase in delayed reactive oxygen species production in tumor is induced and further, the anti-tumor immune response of a host is induced or enhanced. For example, when the composition for inducing tumor immunity of the present invention is applied to radiation therapy, it can also be used as an enhancer of radiation therapy having high safety to humans.

In another aspect, with regard to the composition for inducing tumor immunity of the present invention, by carrying out a combination of ALA administration and irradiation several times, in addition to direct cytotoxic effects provided by irradiation, even after the irradiation, the ALA contributes to induction of long-term immunological effects, such as an increase in delayed reactive oxygen species production in the tumor and induction of tumor cytotoxic M1 macrophages, thereby efficiently inhibiting the growth of the tumor.

In a further aspect, the present inventors have surprisingly found that, when a radiation that is combined with ALA is applied at low energy several times, the effect of the ALA to sensitize radiation therapy is significantly enhanced, in compared to a case where the radiation is applied once at high energy. That is to say, when the composition for inducing tumor immunity of the present invention is combined with divisional application of radiation, it can increase therapeutic effects and can reduce a total dose for obtaining the same effects, in comparison to radiation therapy in which radiation is applied once at the same dose as in the case of divisional application.

That is, the present invention is an extremely important invention in the medical field.

### BRIEF DESCRIPTION OF DRAWINGS

FIG. 1 shows representative images of the subcutaneous tumors of rats after surgical exposure (FIG. 1A to FIG. 1D), and the results of a high performance liquid chromatography (HPLC) analysis showing incorporation of PpIX in tumor (FIG. 1E). FIG. 1A and FIG. 1B each show the tumor of a control group, and FIG. 1C and FIG. 1D each show the tumor of an ALA administration group. FIG. 1A and FIG. 1C each show the tumor in a bright field. FIG. 1B and FIG. 1D each show the fluorescence image of tumor irradiated with a light at 410 nm. In FIG. 1E, the reference character ND indicates “not detected.”

FIG. 2 shows a representative image of the subcutaneous tumors of rats on 16th day after the treatment (FIG. 2A), and the growth curves of subcutaneous tumors (FIG. 2B). The hair covering the skin was removed. After the subcutaneous tumor had grown to a size of 6 to 8 mm in diameter, the rats were randomly divided into four groups, and the tumors were then treated.

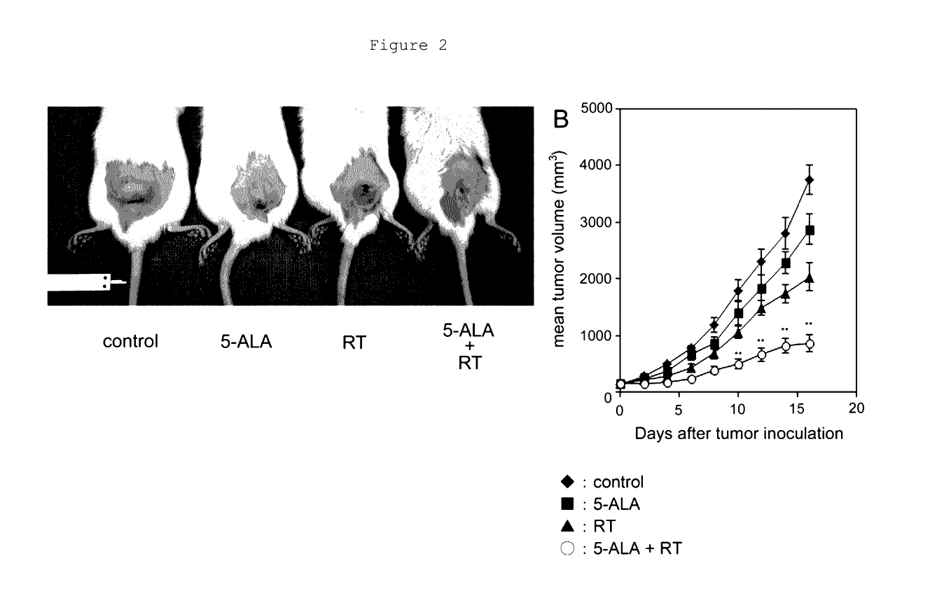

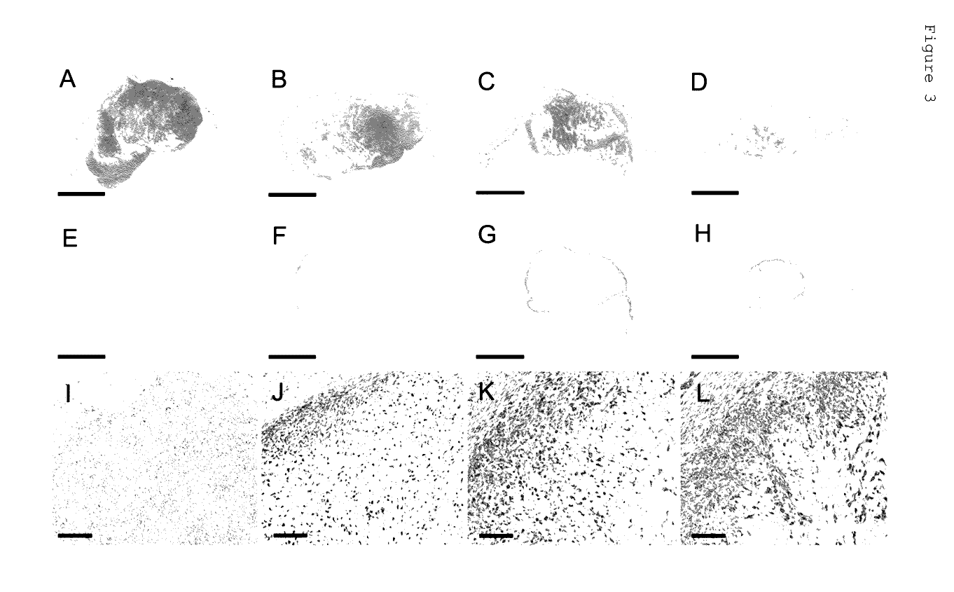

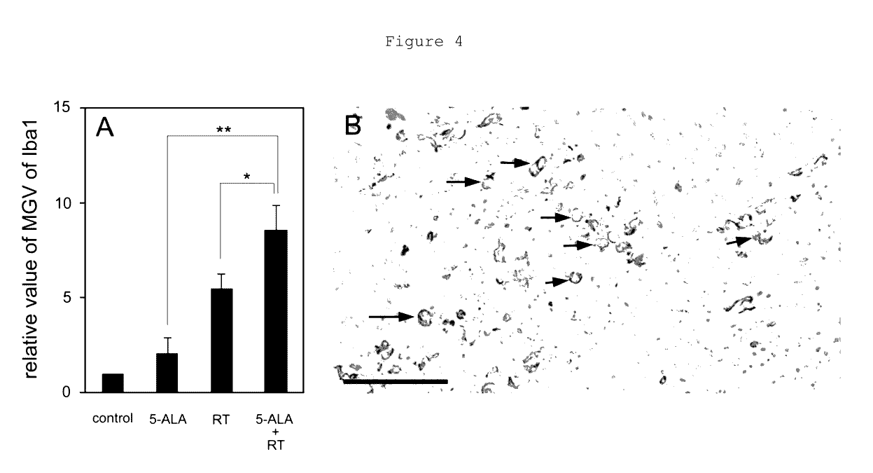

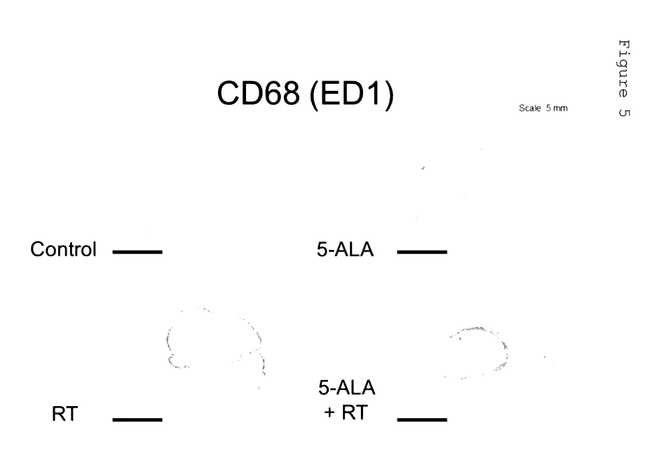

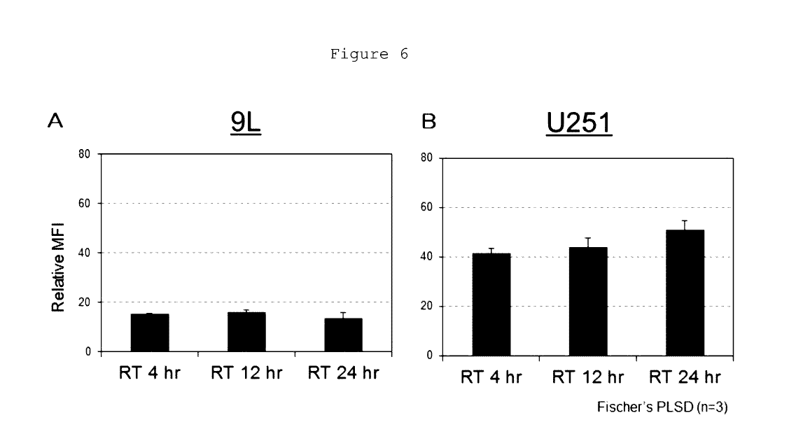

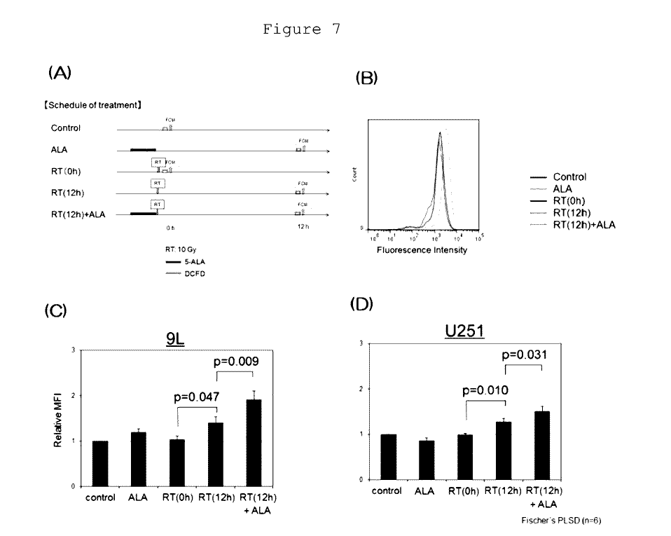

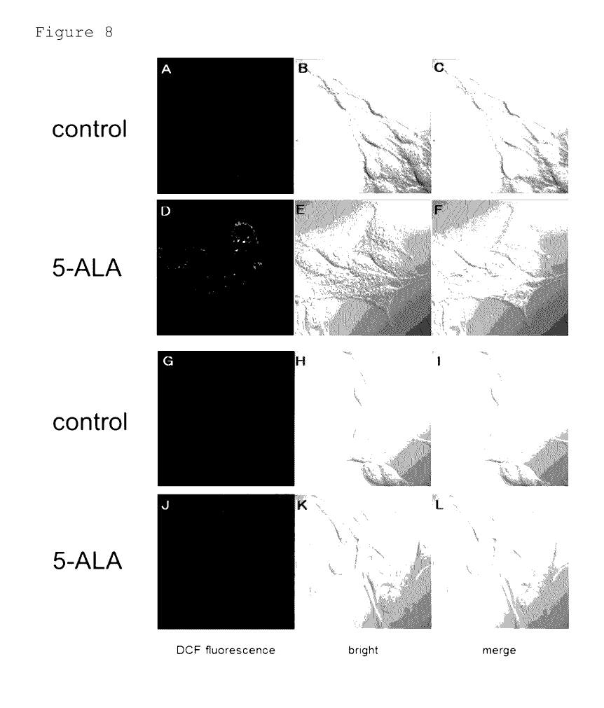

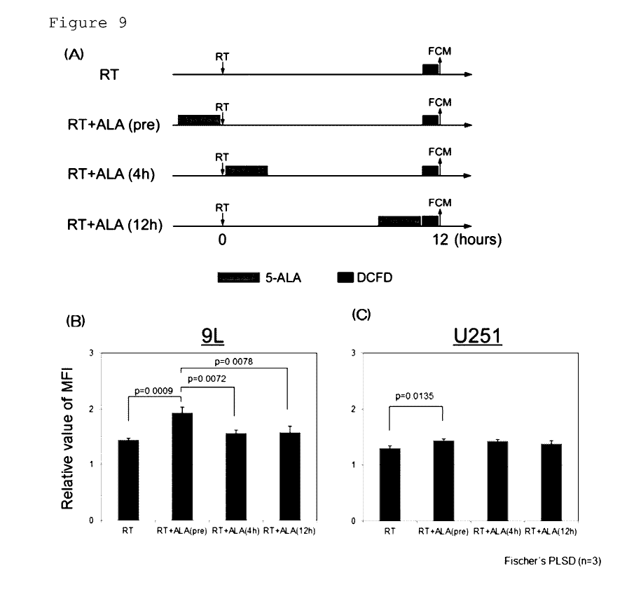

In FIG. 2B, individual reference characters have the following meanings:

control=control group (n=5);
5-ALA=ALA multiple-dose administration group (n=5);
RT=ionizing radiation multiple-dose application group (n=7); and
5-ALA+RT=ALA multiple-dose administration+ionizing radiation multiple-dose application group (n=7). Each dot indicates a mean value. The reference character ** indicates p<0.01, and the mean tumor volume of the ionizing radiation multiple-dose application group was compared with the mean tumor volume of the ALA multiple-dose administration+ionizing radiation multiple-dose application group.

FIG. 3 shows the pathological findings of the subcutaneous tumors of rats 16 hours after the treatment.

FIG. 3A, FIG. 3E, and FIG. 3I: the control group;

FIG. 3B, FIG. 3F, and FIG. 3J: the ALA multiple-dose administration group;

FIG. 3C, FIG. 3G, and FIG. 3K: the ionizing radiation multiple-dose application group; and

FIG. 3D, FIG. 3H, and FIG. 3L: the ALA multiple-dose administration+ionizing radiation multiple-dose application group.

FIG. 3A to FIG. 3D show the results of hematoxylin and eosin staining (HE staining). FIG. 3E to FIG. 3L show the results of Iba1 staining. Regarding each group, the portions stained with Iba1 (FIG. 3E to FIG. 3H) correspond to the portions stained with HE. An enlarged view of the surface of a subcutaneous tumor, which was stained with Iba1, is shown in each of FIG. 3I to FIG. 3L. The scale bar: 5 mm (FIG. 3A to FIG. 3H); 200 μm (FIG. 3I to FIG. 3L).

FIG. 4 shows the relative intensity of the mean gray value (MGV) of Iba1, which was measured by a microdensitometry analysis (FIG. 4A), and representative pathological findings of a boundary zone between coagulation necrosis and surviving tumor cells in a subcutaneous tumor, in the ALA multiple-dose administration+ionizing radiation multiple-dose application group (FIG. 4B; Iba1 staining). control=control group;

5-ALA=ALA multiple-dose administration group;
RT=ionizing radiation multiple-dose application group; and
5-ALA+RT=ALA multiple-dose administration+ionizing radiation multiple-dose application group.
In FIG. 4A, the reference character * indicates p<0.05, and the reference character ** indicates p<0.01. The scale bar: 120 μm (FIG. 4B).

FIG. 5 shows the results obtained by immunostaining the tumor samples used in FIG. 3 with an anti-CD68 antibody.

FIG. 6 shows a change over time in 5-ALA-induced PpIX fluorescence after application of ionizing radiation.

FIG. 7 shows the evaluation of cellular ROS values after application of ionizing radiation. FIG. 7A shows the schedule of treatment in each group. FIG. 7B shows the flow cytometric data of cellular ROS values in 9L cells. FIG. 7C shows the relative MFI of cellular ROS values in 9L cells, after application of ionizing radiation. FIG. 7D shows the relative MFI of cellular ROS values in U251 cells, after application of ionizing radiation.

FIG. 8 shows visualization of cellular ROS 12 hours after application of ionizing radiation, in 9L cells (FIG. 8A-F) and in U251 cells (FIG. 8G-L). In FIGS. 8D-F and J-L, the cells were incubated with 1 mM 5-ALA for 4 hours, and ionizing radiation was then applied to the resulting cells. Twenty hours after application of the ionizing radiation, all of the cells were treated with 10 μM DCFD, so that cellular ROS was specified. The control cells (control) shown in FIGS. 8A-C and G-I were not pre-treated with 5-ALA before application of the ionizing radiation.

FIG. 9 shows the influence of a difference in the timing of 5-ALA treatment upon cellular ROS production 12 hours after application of the ionizing radiation, in 9L cells and U251 cells. FIG. 9A shows the schedule of different timings of 5-ALA treatments. FIG. 9B shows the relative MFI value of each group in a case where 9L cells were used. FIG. 9C shows the relative MFI value of each group in a case where U251 cells were used.

### DESCRIPTION OF EMBODIMENTS

In the present description, the term “ALAS” is used to mean ALA, or a derivative thereof, or their salts.

In the present description, the term “ALA” is used to mean 5-aminolevulinic acid. ALA is also referred to as δ-aminolevulinic acid, and it is one type of amino acid.

An example of the ALA derivative is the compound represented by the above described formula (I). In the formula (I), R¹ represents a hydrogen atom or an acyl group, and R² represents a hydrogen atom, a linear or branched alkyl group, a cycloalkyl group, an aryl group, or an aralkyl group. It is to be noted that, in the formula (I), ALA corresponds to a case where R¹ and R² each represent a hydrogen atom.

ALAs may act as an active ingredient in a living body, in the form of the ALA of the formula (I) or a derivative thereof. In order to increase solubility, ALAs can be administered in the form of various types of salts, esters, or prodrugs (precursors) decomposed by enzymes existing in a living body, depending on the administration form thereof.

Examples of the acyl group represented by R¹ in the formula (I) include: linear or branched alkanoyl groups containing 1 to 8 carbon atoms, such as a formyl, acetyl, propionyl, butyryl, isobutyryl, valeryl, isovaleryl, pivaloyl, hexanoyl, octanoyl, or benzylcarbonyl group; and aroyl groups containing 7 to 14 carbon atoms, such as a benzoyl, 1-naphthoyl, or 2-naphthoyl group.

Examples of the alkyl group represented by R² in the formula (I) include: linear or branched alkyl groups containing 1 to 8 carbon atoms, such as a methyl, ethyl, propyl, isopropyl, butyl, isobutyl, sec-butyl, tert-butyl, pentyl, isopentyl, neopentyl, hexyl, heptyl, or octyl group.

Examples of the cycloalkyl group represented by R² in the formula (I) include: saturated, or partially unsaturated cycloalkyl groups containing 3 to 8 carbon atoms, such as a cyclopropyl, cyclobutyl, cyclopentyl, cyclohexyl, cycloheptyl, cyclooctyl, cyclododecyl, or 1-cyclohexenyl group.

Examples of the aryl group represented by R² in the formula (I) include: aryl groups containing 6 to 14 carbon atoms, such as a phenyl, naphthyl, anthryl, or phenanthryl group.

Examples of the aralkyl group represented by R² in the formula (I) include groups, the aryl portions of which are the same as those of the aforementioned aryl groups, and the alkyl portions of which are the same as those of the aforementioned alkyl groups. Specific examples of such as an aralkyl group include aralkyl groups containing 7 to 15 carbon atoms, such as a benzyl, phenethyl, phenylpropyl, phenylbutyl, benzhydryl, trityl, naphthylmethyl, or naphthylethyl group.

A preferred ALA derivative can be a compound in which R¹ is a formyl, acetyl, propionyl, or butyryl group. Another preferred ALA derivative can be a compound in which the above R² is a methyl, ethyl, propyl, butyl, or pentyl group. A further preferred ALA derivative can be a compound in which the combination of the above R¹ and R² is (formyl and methyl), (acetyl and methyl), (propionyl and methyl), (butyryl and methyl), (formyl and ethyl), (acetyl and ethyl), (propionyl and ethyl), or (butyryl and ethyl).

Among the ALAs, examples of the salts of the ALA or a derivative thereof include pharmacologically acceptable acid-added salts, metal salts, ammonium salts, and organic amine-added salts. Examples of the acid-added salts include: various inorganic acid salts such as hydrochloride, hydrobromide, hydroiodide, phosphate, nitrate, or sulfate; and various organic acid-added salts such as formate, acetate, propionate, toluenesulfonate, succinate, oxalate, lactate, tartrate, glycolate, methanesulfonate, butyrate, valerate, citrate, fumarate, maleate, or malate. Examples of the metal salts include: various alkaline metal salts such as lithium salt, sodium salt, or potassium salt; various alkaline-earth metal salts such as magnesium salt or calcium salt; and various metal salts such as aluminum or zinc. Examples of the ammonium salts include alkyl ammonium salts such as ammonium salt or tetramethyl ammonium salt. Examples of the organic amine salts include various salts such as triethylamine salt, piperidine salt, morpholine salt, or toluidine salt. These salts can also be used in the form of a solution, upon use.

Among the aforementioned ALAs, examples of the most desirable ALA include: ALA; various types of esters, such as ALA methyl ester, ALA ethyl ester, ALA propyl ester, ALA butyl ester, or ALA pentyl ester; and their hydrochloride, phosphate, and sulfate. Among others, examples of particularly preferred ALA include ALA hydrochloride and ALA phosphate.

The above described ALAs can be produced according to known methods, such as chemical synthesis, production using microorganisms, and production using enzymes. In addition, the above described ALAs may form hydrates or solvates. Moreover, the ALAs can be used alone or in combination of two or more types, as appropriate.

When the above described ALAs are prepared in the form of an aqueous solution, it is necessary to pay attention not to convert the aqueous solution to an alkaline solution, so as to prevent decomposition of the ALAs. If the aqueous solution is converted to an alkaline solution, the decomposition of the ALAs can be prevented by removing enzymes.

In the present description, tumor immunity is used to typically mean an immune mechanism to attack tumor cells (cancer cells). The immune mechanism of a host is able to recognize tumor cells and eliminate them. It has been known that both an innate immune system and an acquired immune system are involved in the tumor immunity, and that the tumor immunity is able to suppress the growth of tumor. The present inventors have found from an in vivo experiment that the ALA itself is able to induce or enhance the anti-tumor immune response of a host even under an environment where there is no light irradiation to tumor. Accordingly, the composition for inducing tumor immunity of the present invention, or the preventive or therapeutic medicament of the present invention is able to induce an immune mechanism to attack tumor cells and is able to contribute to suppression of tumor growth, in a subject (preferably, a human), alone or in combination with, for example, radiation therapy, photodynamic therapy, surgical resection of tumor, or anti-cancer therapy. In a preferred aspect, the composition of the present invention can function as an enhancer of radiation therapy.

Cytotoxic T cells (CTL) are cells playing a main role in tumor immunity, and are responsible for the removal of virus-infected cells, tumor cells, and the like. Cells, which are judged as “non-self” cells (foreign organisms) by CTL, are induced to cell death (apoptosis). Differentiation of CTL is induced by antigen presentation from dendritic cells. The dendritic cells have been known to act as antigen-presenting cells. After the dendritic cell has incorporated an antigen therein, it decomposes it, and then presents a fragment thereof on the cell surface thereof. Helper T cells, which have received such antigen presentation, activate and release cytokines, so as to promote differentiation and/or growth of CTL. In addition, it has also been known that not only an adaptive immune system, but also an innate immune system, such as NK cells, NKT cells, macrophages or granulocytes, is associated with tumor immunity, and that such an innate immune system suppresses the growth of tumor. Moreover, for example, a tumor antigen (particularly, a tumor-specific antigen; an antigen which is not expressed in normal cells but is expressed in tumor cells), etc. is expressed in tumor cells, and such a tumor antigen is recognized by CTL or the like, and thereby, tumor cells can be eliminated.

The action mechanism of ALAs regarding induction of tumor immunity has not been completely elucidated. For instance, by inducing tumor cytotoxic M1 macrophages to tumor cells or activating such cytotoxic M1 macrophages, ALAs can contribute to suppression of the growth of tumor.

In the present description, cancer is preferably malignant neoplasm, but the type of the cancer is not limited. Examples of the cancer include primary or metastatic, and invasive or non-invasive cancers or sarcomas, such as brain tumor, spinal cord tumor, maxillary sinus carcinoma, pancreatic ductal adenocarcinoma, carcinoma of gingiva, tongue cancer, lip cancer, nasopharyngeal carcinoma, oropharynx carcinoma, hypopharyngeal carcinoma, laryngeal cancer, thyroid cancer, parathyroid cancer, lung cancer, pleural tumor, carcinomatous peritonitis, carcinomatous pleurisy, esophageal cancer, stomach cancer, colon cancer, bile duct cancer, gallbladder cancer, pancreatic cancer, liver cancer, kidney cancer, bladder cancer, prostate cancer, penile cancer, testicular tumor, adrenal cancer, cervical cancer, uterine cancer, vaginal cancer, vulvar cancer, ovary cancer, bone tumor, breast cancer, skin cancer, melanoma, basal cell carcinoma, lymphoma, Hodgkin's disease, plasmacytoma, osteosarcoma, chondrosarcoma, liposarcoma, rhabdomyosarcoma, and fibrosarcoma. The cancer is particularly preferably brain tumor. In addition, the cancer is preferably solid cancer. The tumor cells (cancer cells) in the present invention may be derived from the aforementioned cancers or sarcomas.

The administration route of the composition for inducing tumor immunity of the present invention or the preventive or therapeutic medicament of the present invention may be either systemic administration or local administration. Examples of the administration route include, but are not limited to, oral administration including sublingual administration; and parenteral administration, such as nasal administration, inhalation administration, direct administration to subject tissues or organs using a catheter, intravenous administration including infusion, transdermal administration including poultice or the like, and administration involving force enteral nutrition methods using a suppository, or a nasogastric tube, nasal enteric tube, gastrostomy tube or intestinal fistula tube.

The dosage form of the composition for inducing tumor immunity of the present invention, or the dosage form of each component comprised in the preventive or therapeutic medicament of the present invention (e.g. a compounding agent or a kit), is not limited, and it may be determined, as appropriate, depending on the above described administration route. Examples of the dosage form include an injection, an infusion, a tablet, a capsule, a fine granule agent, a powdered agent, a liquid agent, a water agent prepared by dissolution in syrup or the like, a poultice, and a suppository. The dosage form of the composition for inducing tumor immunity of the present invention, or each component comprised in the preventive or therapeutic medicament of the present invention, is not limited to medicinal use, but it can also be processed into the form of supplements, such as a tablet or a capsule. In addition, in particular, for aged people, infants, and the like, who have difficulty in swallowing medicines, the form of a disintegrating tablet that exhibits prompt disintegration in mouth, or a liquid agent suitable for nasogastric administration is preferable.

In order to prepare the composition for inducing tumor immunity of the present invention or the preventive or therapeutic medicament of the present invention (e.g. a compounding agent or a kit), for example, a pharmacologically acceptable carrier, an excipient, a diluent, an additive, a disintegrator, a binder, a coating agent, a lubricant, a sliding agent, a lubricating agent, a flavoring agent, a sweetener, a solubilizer, a solvent, a gelling agent, and a nutrient may be added, as necessary. The use of these agents may have influence on the absorbability and blood concentration of the composition for inducing tumor immunity of the present invention or the preventive or therapeutic medicament of the present invention, and may also provide a change in the pharmacokinetics of the present composition or medicament. Specific examples of the additive include water, normal saline, animal fat and oil, vegetable oil, lactose, starch, gelatin, crystalline cellulose, gum, talc, magnesium stearate, hydroxypropyl cellulose, polyalkylene glycol, polyvinyl alcohol, and glycerin.

The preventive or therapeutic medicament of the present invention means a combination of two or more substances or compositions, wherein the aspect of the combination is not limited.

Needless to say, to the preventive or therapeutic medicament of the present invention, any given other components, such as other medicinal components, nutrients and carriers, can be added, as necessary. Examples of such any given components, which can be added to the preventive or therapeutic medicament of the present invention, include various types of mixing components for preparations, including a pharmaceutically acceptable ordinary carrier, binder, stabilizer, solvent, dispersion medium, thickener, excipient, diluent, pH buffer, disintegrator, solubilizer, solubilizing agent, and tonicity agent, such as, for example, crystalline cellulose, gelatin, lactose, starch, magnesium stearate, talc, vegetable and animal fats, oil and fat, gum, or polyalkylene glycol.

The administration subject of the composition for inducing tumor immunity of the present invention or the preventive or therapeutic medicament of the present invention (e.g. a compounding agent or a kit) is typically a mammal (preferably, a human), and further, non-human animals such as pet animals, experimental animals and livestock animals are also included in the administration subject. In addition, in a case where it is not preferable, a human may be excluded from the subject. The above described subject may include, not only a subject which is affected with cancer, but also a subject which is in danger of developing cancer or having already had cancer.

With regard to the dose, timing, administration frequency, administration period and the like of the composition for inducing tumor immunity of the present invention or the preventive or therapeutic medicament of the present invention (e.g. a compounding agent or a kit), these conditions are not particularly limited, as long as the present composition or medicament can be used in an effective amount in which tumor immunity can be induced in a subject, and thus, the aforementioned conditions can be determined by a person skilled in the art, as appropriate. When the subject is a human for example, the aforementioned conditions may be different, depending on the age, body weight, symptoms, etc. of a cancer patient.

With regard to the dose of ALAs to a human, when ALA hydrochloride is applied to a human via oral administration, the applied dose may be 1 mg to 1,000 mg, preferably 5 mg to 100 mg, more preferably 10 mg to 30 mg, and further preferably 15 mg to 25 mg per kg of body weight, relative to ALA.

The timing of administration of ALAs to a human may be changed, as appropriate, depending on therapeutic purpose. For instance, in a case where ALA administration is combined with radiation therapy, the ALA may be administered 2 to 12 hours before, preferably 3 to 9 hours before, and more preferably 3 to 6 hours before the irradiation.

The administration frequency of ALAs may be a single-dose or multiple-dose administration per day, or continuous administration using infusion or the like. For example, in the case of brachytherapy that is a method of detaining a radiation source (radioisotope) near a tumor, the administration frequency of ALAs may be a single-dose or multiple-dose administration per day, or continuous administration using infusion or the like.

The administration period of ALAs can be determined by pharmacologists or clinicians in the present technical field, for example, according to known methods, based on various clinical indexes, while considering the symptoms and the like of the subject.

The present inventors have discovered that the growth of tumor is inhibited even by repetitive administration of ALA alone. Accordingly, ALAs are preferably administered repeatedly at least two or more times (e.g. two times, three times, four times, or more times).

Moreover, in the case of radiation therapy, radiation is preferably applied once or several times to tumor cells in a subject. The present inventors have surprisingly found that when a radiation that is combined with ALA administration is applied at low energy several times, the sensitizing effects of the ALA in radiation therapy are significantly enhanced, in compared to a case where the radiation is applied once at high energy. Therefore, it is preferable that the radiation be repeatedly applied to the subject at least two or more times (e.g. two times, three times, four times, or more times).

For example, as a radiation used herein, electron beam such as X-rays or γ-rays, and particle beam such as heavy ion beam, proton beam or neutron beam may be used. The exposure dose of the radiation, irradiation timing, irradiation frequency and the like may be changed, as appropriate, depending on therapeutic purpose, while also taking into consideration a combination with administration of ALAs, etc. Examples of the radiation therapy applied herein may include external irradiation, sealed brachytherapy, and unsealed brachytherapy. As an example, in the case of using X-rays, the radiation may be applied at a dose of 1 Gy to 20 Gy, preferably 1.5 Gy to 3 Gy, and more preferably 1.8 Gy to 2 Gy, per human per irradiation. A person skilled in the art can change the applied dose and the like, as appropriate, depending on a radiation source, an irradiated site, irradiation purpose, etc. For example, in the case of radiation therapy performed on the central nerve, the dose may be 1.8 to 2 Gy/day. For example, when radiation therapy is performed for the purpose of alleviation of bone metastasis, a single dose may be further increased. Moreover, in the case of stereotactic irradiation (which is irradiation with external radiations from multiple directions in a short time), a single dose may be approximately 20 Gy. While paying attention not to cause side effects to a living body, the radiation is preferably applied at a low dose, dividedly over several irradiations.

In the present description, the term “repeat” is intended to simply mean that a specific action (e.g. administration of ALA, application of radiation, etc.) is repeatedly carried out. Needless to say, the applied dose of ALA, the applied dose of radiation, administration interval, irradiation interval, and the like, in each action, may be identical to or different from one another.

Furthermore, by appropriately combining the multiple-dose administration of ALAs (e.g. two times, three times, four times, or more times) with application of multiple-dose radiation (e.g. two times, three times, four times, or more times), further synergic effects can be preferably expected, regarding cancer therapy. Since ALA is metabolized in a living body and is then discharged from the body within 48 hours, it is clinically highly safe. Accordingly, the combination of the multiple-dose administration of ALAs with application of multiple-dose radiation is advantageous in that it further activates tumor immunity, while ensuring safety to a living body. Further, the combination of the administration of ALAs with divisional application of radiation is advantageous in that it can increase therapeutic effects and can reduce a total dose for obtaining the same effects, in comparison to radiation therapy in which radiation is applied once at the same dose as in the case of divisional application.

For example, by applying radiation after administration of ALAs, then administering the ALAs again, and then applying radiation, it becomes possible to kill tumor cells existing in a deep part of tumor, which could not be achieved by the initial ALA administration and irradiation. When ALAs are administered by multiple-dose administration, irradiation may be carried out several times between the administrations.

Otherwise, in a case where, clinically, therapeutic effects can be continued (effects can appear) for a certain period of time (for example, 2 to 3 months) after completion of the irradiation (treatment), ALAs may be administered for a certain period of time (for example, 2 to 3 months) after completion of the radiation therapy.

Further, for example, surgical resection of tumor, anti-cancer therapy or photodynamic therapy has been firstly performed to kill a majority of cancer cells, and thereafter, ALA administration, and in some cases, irradiation may be carried out, once or several times, on remaining tumor cells, or on tissues in which tumor cells are likely to remain. By appropriately carrying out such a combination therapy, as desired, the remaining cancer cells are killed, or the recurrence or metastasis of cancer can be prevented.

In one aspect, the composition for inducing tumor immunity of the present invention or the preventive or therapeutic medicament of the present invention may comprise a metal-containing compound, as long as such a metal-containing compound does not give unacceptable adverse effects to a living body and the like and the purpose and object of the present invention can be achieved. The metal part in such a metal-containing compound is not limited, and examples of the metal part include iron, magnesium, zinc, nickel, vanadium, cobalt, copper, chromium, and molybdenum. In light of the purpose and object of the present invention, a person skilled in the art can select a suitable dose of metal-containing compound, as appropriate, and can administer it together with ALAs.

For instance, the composition for inducing tumor immunity of the present invention and a metal-containing compound (e.g. an iron compound) can be administered, in the form of a composition comprising ALAs and a metal-containing compound, or each alone. When the present composition and the metal-containing compound are administered each alone, they may be administered simultaneously or sequentially. The term “simultaneous” is used herein to mean, not only that administrations are carried out at the same time as each other, but also that although administration of the ALAs and administration of the metal-containing compound are not carried out at the same time as each other, the administrations are carried out without having a considerable interval between them, such that the administrations can give additive effects, and preferably synergic effects on a living body.

For example, when the iron-containing compound is an iron compound, the compound may be either an organic salt or an inorganic salt. Examples of the inorganic salt include ferric chloride, iron sesquioxide, iron sulfate, and ferrous pyrophosphate. Examples of the organic salt include carboxylates, for example, hydroxycarboxylates that are citrates, such as ferrous citrate, iron sodium citrate, ferrous sodium citrate, or iron ammonium citrate; organic acid salts such as ferric pyrophosphate, iron lactate, ferrous gluconate, sodium iron diethylenetriamine pentaacetate, ammonium iron diethylenetriamine pentaacetate, sodium iron ethylenediamine tetraacetate, ammonium iron ethylenediamine tetraacetate, sodium iron dicarboxymethylglutamate, ammonium iron dicarboxymethylglutamate, ferrous fumarate, iron acetate, iron oxalate, ferrous succinate, or sodium iron succinate citrate; and heme iron, dextran iron, triethylenetetramine iron, lactoferrin iron, transferrin iron, sodium iron chlorophyllin, ferritin iron, saccharated iron oxide, and ferrous glycine sulphate. Among these, ferrous sodium citrate and iron sodium citrate are preferable.

The above described iron compounds may be used alone or in combination of two or more types. With regard to the dose of such an iron compound, the iron compound may be used at a molar ratio of 0.01 to 100, more preferably 0.05 to 10, and more preferably 0.1 to 8, based on the dose of ALAs (relative to ALA). The method of administering the ALAs may be identical to or different from the method of administering the iron compound. For example, the composition for inducing tumor immunity of the present invention or the preventive or therapeutic medicament of the present invention may comprise the iron compound as well as the ALAs. Otherwise, apart from administration of the composition for inducing tumor immunity of the present invention or the preventive or therapeutic medicament of the present invention, the iron compound may be administered to a subject as a combined use, separately. Needless to say, as in the case of the ALAs, the dose of the iron compound applied to a subject, the timing of administration, administration frequency, administration period, etc. can be determined by a person skilled in the art, as appropriate, while taking into consideration the properties of the iron compound and the type thereof.

In addition, the anticancer agent used in the present invention is not particularly limited, as long as it is an anticancer agent known to a person skilled in the art. Examples of such an anticancer agent include: alkylating agents (nitrogen mustards (cyclophosphamide, etc.); nitrourea, etc.); platinum compounds (cisplatin, etc.); antimetabolic agents (5-fluorouracil, etc.); antifolates (dihydropteroate synthase inhibitor; dihydrofolate reductase inhibitor, etc.); pyrimidine metabolism inhibitors; purine metabolism inhibitors; ribonucleotide reductase inhibitors; nucleotide analogs (purine analog, pyrimidine analog, etc.); topoisomerase inhibitors (type I topoisomerase inhibiting agent; type II topoisomerase inhibiting agent, etc.); microtubule inhibitors (microtubule polymerization inhibitor; microtubule depolymerization inhibitor, etc.); antitumor antibiotics; endocrine therapy; vaccine therapy; and molecularly-subjected drugs (low molecular weight pharmaceutical products (tyrosine kinase inhibiting agents; Raf kinase inhibitors; proteasome inhibiting agents, etc.); antibody drugs, etc.). These anticancer agents are considered to have an action mechanism that is fundamentally different from those of ALA or radiation therapy. Accordingly, it can be anticipated that the anticancer agents have additive effects, and in some cases, synergic effects.

Such an anticancer agent may be combined with the composition for inducing tumor immunity of the present invention or the preventive or therapeutic medicament of the present invention, and the two agents may be administered to a subject, simultaneously or sequentially. The method of administering the ALAs may be identical to or different from the method of administering the anticancer agent. For example, the ALAs may be administered to a subject via oral administration, and the anticancer agent may be administered to the subject via intravenous administration. Needless to say, the dose of the anticancer agent applied to a subject, the timing of administration, administration frequency, administration period, etc. can be determined by a person skilled in the art, as appropriate, while taking into consideration the properties of the anticancer agent and the type thereof.

In one aspect, the ALAs and the anticancer agent may be used, as a preventive or therapeutic medicament(s), in the form of a composition comprising the ALAs and the anticancer agent (compounding agent), or as different kits. Otherwise, they may also be administered to a subject each alone. When the ALAs and the anticancer agent are administered to a subject each alone, they may be administered simultaneously or sequentially. Herein, the term “simultaneous” is used to mean, not only that administrations are carried out at the same time as each other, but also that although administration of the ALA and administration of the anticancer agent are not carried out at the same time as each other, the administrations are carried out without having a considerable interval between them, such that the administrations can give additive effects, and preferably synergic effects.

Moreover, since the composition for inducing tumor immunity of the present invention or the preventive or therapeutic medicament of the present invention has the effect of enhancing radiation therapy, by combining the ALAs with a known radiosensitizer (e.g. a nitroimidazole-based radiosensitizer, etc.) or with hyperbaric oxygen therapy or the like, additive effects, and preferably, synergic effects can be expected. Likewise, the ALAs may also be combined with, for example, a radiation side effect suppressor such as zyûzen-taihotô. Furthermore, one or two or more types of existing agents for preventing and/or treating radiation injury, such as cytokine, nitroprusside, lactoferrin, 6,10,14,18-tetramethyl-5,9,13,17-nonadecatetraen-2-one, a pyrazolone derivative, growth factors SCF, IL3, GM-CSF and IL6, (±)-N,N′-propylene dinicotinamide, 13-oxygermylpropionic acid, β-lapachone, a phosphorus derivative of alkaloid, α-D-glucopyranosyl-(1→2)-L-ascorbic acid, amifostine, interferon, a non-steroidal anti-inflammatory agent (NSAIDS), a new quinolone preparation, and a statin preparation, which act to promote hematopoiesis, may be combined and used with the composition for inducing tumor immunity of the present invention or the preventive or therapeutic medicament of the present invention.

Needless to say, the dose of such an additional component applied to a subject, the timing of administration, administration frequency, administration period, etc. can be determined by a person skilled in the art, as appropriate, while taking into consideration the properties of the component and the type thereof.

When ALAs, an anticancer agent, and radiation therapy are combined with one another to prevent or treat cancer, for example, the anticancer agent may be administered to a subject, the ALAs may be then administered thereto, and a cycle of applying a radiation thereto once or several times may be then carried out once or several times. Alternatively, after the ALAs have been administered to a subject, the anticancer agent may be administered thereto, and thereafter, a cycle of applying a radiation thereto once or several times may be then carried out once or several times. Alternatively, after the ALAs have been administered to a subject, a cycle of applying a radiation thereto once or several times may be carried out once or several times, and thereafter, the anticancer agent may be then administered to the subject once or several times. Likewise, an iron compound, a known radiosensitizer, and the like may be combined with one another, as appropriate, and they may then be administered to a subject.

Thus, needless to say, in addition to the ALA single-dose or multiple-dose administration, any given combinations of single-dose or multiple-dose administration of the anticancer agent, single-dose or multiple-dose application of radiation, and the like, are included in the scope of the present invention.

The composition for inducing tumor immunity of the present invention or the preventive or therapeutic medicament of the present invention can also be used as a pharmaceutical product, a quasi-drug, a cosmetic product, food and beverage, a feed, a bait, or pet food.

With regard to the method for inducing tumor immunity in a subject affected with cancer of the present invention, needless to say, the dose of each component, the timing of administration, administration frequency, administration period, the dose of radiation, irradiation frequency, and the order or combination of individual therapies, etc. are determined in the same manner as that described above.

### [Other Effects of the Present Invention]

By using ALAs as a radiosensitizer, therapeutic effects on various cancer species can be enhanced. Radiation therapy has been generally used for malignant tumors. On the other hand, this therapy has been concerned about damage on normal tissues. Thus, by the combined use of ALAs with such radiation therapy, it cannot only enhance therapeutic effects, but it can also reduce the irradiation dose, thereby reducing the influence on normal tissues.

In addition, for the treatment of malignant brain tumor, postoperative radiation therapy and chemotherapy have been typically carried out. However, in the case of glioblastoma having the highest malignancy, the life expectancy is only approximately 1 year. In the case of brain tumor, it becomes possible to suppress tumor growth by the combined use of irradiation. However, delayed radiation injury (radiation necrosis, leukoencephalopathy, etc.) to the central nervous system would become problems. These side effects depend on irradiation dose.

The present inventors have found that it becomes possible to significantly suppress tumor growth by combining administration of ALAs with irradiation (divisional irradiation). According to the present invention, it becomes possible to further extend the survival period of cancer patients, and to reduce irradiation dose thereby reducing side effects. On the other hand, it has been reported that PpIX induced from ALAs is accumulated not only in malignant brain tumor, but also in various cancers. Hence, a person skilled in the art can naturally understand that the use of ALAs will have the same effects as those described above on various cancers. Further, ALA has already been widely used in clinical sites, and its safety has also been established. Therefore, the ALAs have extremely high utility values as radiosensitizers (enhancers of radiation therapy).

The terms used in the present description are used to explain specific embodiments, and thus, they are not intended to limit the scope of the invention.

Moreover, the term “comprise” used in the present description is intended to mean that the mentioned matter (a member, a step, an element, a number, etc.) is present, except for a case where the term should be understood in a contextually apparently different way, and thus, the presence of other matters (other members, steps, elements, numbers, etc.) is not excluded.

Unless otherwise specified, all of the terms used herein (including technical terms and scientific terms) have the same meanings as those which are broadly understood by a person skilled in the art in the technical field, to which the present invention pertains. Unless a different definition is clearly specified, the terms used herein should be understood to have meanings which are consistent with the meanings in the present description and the relevant technical field. The terms used herein should not be understood to have ideal or excessively formal meanings.

There is a case where the embodiment of the present invention is described, while referring to a schematic view. In the case of using such a schematic view, there is a case where exaggerated expressions are used in order to make the explanation clear.

The term such as “first . . . ” or “second . . . ” is used to express various elements. It is understood, however, that these elements should not be limited by such terms. These terms are only used to distinguish one element from other elements. Thus, it is understood that it is possible to describe, for example, a first element as a second element, and likewise, to describe the second element as the first element, without deviating from the scope of the present invention, unless it includes a technical contradiction.

In the present description, when the expression “alkanoyl group containing 1 to 8 carbon atoms” is used for example, a person skilled in the art understands that this expression specifically indicates individual alkyl groups containing 1, 2, 3, 4, 5, 6, 7 or 8 carbon atoms.

In the present description, all numerical values used to indicate ingredient contents or numerical value ranges are understood to include the meaning of the term “about,” unless otherwise specified.

All of the disclosures of publications cited in the present description should be incorporated herein by reference. A person skilled in the art uses and understands the relevant disclosure in those prior art publications as a part of the present description, along with the context thereof, without deviating from the gist and scope of the present invention.

Hereinafter, the present invention will be described more in detail, referring to examples. However, the present invention can be realized in various aspects, and thus, it must not be interpreted that the scope of the present invention is limited to the following examples.

### EXAMPLES

### Example 1

### Accumulation of Macrophages in Tumor Tissue Site by Administration of ALA

### Materials and Methods

### [Chemical Substances]

ALA was purchased from Cosmo Bio K.K. (Tokyo, Japan), and was then dissolved in a concentration of 100 mg/ml in a phosphate buffered saline (PBS). Using 10 N sodium hydroxide (NaOH), the pH of the solution was adjusted to be pH 6.0 to 6.3. The solution was used within 10 minutes after the preparation thereof.

### [Brain Tumor Cell Lines and Animals]

As a brain tumor cell line, there were used Inbred Fischer rat-derived 9L gliosarcoma cells (Benda P, Someda K, Messer J, Sweet W H: Morphological and immunochemical studies of rat glial tumors and clonal strains propagated in culture. J Neurosurg 34: 310-23, 1971; Schmidek H H, Nielsen S L, Schiller A L, Messer J: Morphological studies of rat brain tumors induced by N-nitrosomethylurea. J Neurosurg 34: 335-40, 1971.), which have been widely used as experimental models of gliosarcoma in inbred rat strains. Since the present cells have both appearances of glioblastoma and sarcoma, they are named as “9L gliosarcoma cells.” Such 9L gliosarcoma cells were acquired from Dr. Tokuyama, Hamamatsu University School of Medicine, Japan. Before use, the cells were cultured in an RPMI1640 medium containing 10% fetal calf serum (FCS) for several days.

As experimental animals, inbred male Fischer 344 rats (8 week old) (SLC, Inc. (Hamamatsu, Japan)) were used and were inoculated with 9L gliosarcoma cells. The inoculation was carried out in accordance with the procedures described in the following publications: Yamamoto J, Hirano T, Li S, Koide M, Kohno E, Inenaga C, et al.: Selective accumulation and strong photodynamic effects of a new photosensitizer, ATX-S10.Na (II), in experimental malignant glioma. Int J Oncol 27: 1207-13, 2005; Yamamoto J, Yamamoto S, Hirano T, Li S, Koide M, Kohno E, et al.: Monitoring of singlet oxygen is useful for predicting the photodynamic effects in the treatment for experimental glioma. Clin Cancer Res 12: 7132-9, 2006. In short, 9L gliosarcoma cells (1×10⁶ cells) were transplanted into the skin on the dorsal side of each Fischer 344 rat, so as to produce a rat subcutaneous tumor model.

### [Evaluation of Accumulation of ALA-Induced PpIX in Rat Subcutaneous Tumor Model]

The present inventors have first conducted a high performance liquid chromatography (HPLC) analysis and fluorescence observation, so that they have confirmed accumulation of PpIX induced by ALA, in the rat subcutaneous tumor models as produced above.

Specifically, after the transplanted tumor had grown to a size of 1 cm in diameter, 100 mg/kg body weight of ALA was injected into the caudal vein of each rat (i.v.). Three hours later, the rat was anesthetized, and a tumor portion, excluding a portion covering the dorsal skin, was excised from the rat. The tumor portion was immediately subjected to a freezing treatment using dry ice, and the resultant was then preserved at −80° C. in a dark place.

Subsequently, the HPLC analysis was carried out in accordance with the procedures described in the following publications: Hagiya Y, Fukuhara H, Matsumoto K, Endo Y, Nakajima M, Tanaka T, et al.: Expression levels of PEPT1 and ABCG2 play key roles in 5-aminolevulinic acid (ALA)-induced tumor-specific protoporphyrin IX (PpIX) accumulation in bladder cancer. Photodiagnosis Photodyn Ther 10: 288-95, 2013; Ishizuka M, Hagiya Y, Mizokami Y, Honda K, Tabata K, Kamachi T, et al.: Porphyrins in urine after administration of 5-aminolevulinic acid as a potential tumor marker. Photodiagnosis Photodyn Ther 8: 328-31, 2011.

Specifically, a tumor sample (approximately 1 mm in diameter) was treated with 200 μl of 0.1 M NaOH, and it was then homogenized on ice, using Powermasher II (Assist, Tokyo, Japan). An aliquot (10 μl) of the sample treated with NaOH was picked up, and was then used in Protein Concentration Assay (Quick Start™ Bradford Dye Reagent, Bio-Rad Laboratories, Inc., CA). Moreover, an N-dimethylformamide:isopropanol solution (100:1, v/v) was added to the sample treated with NaOH, in a volume three times larger than that of the sample (150 μl), so that 50 μl of the remaining cell protein was degenerated.

The prepared sample, which had been preserved overnight in a dark place, was treated by procedures, which had been modified from the procedures described in the following publications: Hagiya Y, Fukuhara H, Matsumoto K, Endo Y, Nakajima M, Tanaka T, et al.: Expression levels of PEPT1 and ABCG2 play key roles in 5-aminolevulinic acid (ALA)-induced tumor-specific protoporphyrin IX (PpIX) accumulation in bladder cancer. Photodiagnosis Photodyn Ther 10: 288-95, 2013; Ishizuka M, Hagiya Y, Mizokami Y, Honda K, Tabata K, Kamachi T, et al.: Porphyrins in urine after administration of 5-aminolevulinic acid as a potential tumor marker. Photodiagnosis Photodyn Ther 8: 328-31, 2011. In short, while the temperature was maintained at 40° C., porphyrin was separated using a HPLC system (Type Prominence, Shimadzu, Kyoto, Japan) equipped with a reverse phase C18 column (CAPCELL PAK, C18, SG300, 5 μm, 4.6 mm×250 mm; Shiseido, Tokyo, Japan). As elution solvents, Solvent A (1 M ammonium acetate containing 12.5% acetonitrile, pH 5.2) and Solvent B (50 mM ammonium acetate containing 80% acetonitrile, pH 5.2) were used.

For the elution, Solvent A was used for 5 minutes, then, linear gradient Solvent B (0% to 100%) was used for 25 minutes, and thereafter, Solvent B was used for 10 minutes. The flow rate for elution was maintained at constant, using a fluorospectro-photometer (excited at 404 nm, and detected at 624 nm). From the calibration curve produced using porphyrin as a reference, the porphyrin level in the sample was estimated.

Thereafter, the dorsal skin of a rat under anesthesia, which covered the inoculated tumor, was turned over, and 100 mg/kg body weight of ALA was then intravenously administered. Three hours after the administration, the subcutaneous tumor was observed from the back side of the skin. The bright field of the subcutaneous tumor was photographed with a digital camera (D90, Nikon, Japan), using, as light sources, externally-equipped halogen lamp (C-FID, Nikon, Japan) and a longpass filter. Subsequently, blue-violet light was applied to the subcutaneous tumor (410 nm Light-Emitting Diode Illuminator, SBI Pharma CO., Ltd., Tokyo, Japan), and the tumor image was then photographed with a digital camera, using a longpass filter.

### [Evaluation of In Vivo Radiosensitizing Effect of ALA, Upon Application of Multiple Doses of Ionizing Radiation, Using Rat Subcutaneous Tumor Models]

As described above, 9L gliosarcoma cells were inoculated into inbred Fischer 344 rats. According to the previous studies, it had been demonstrated that the tumor growth of 9L gliosarcoma cells (9L tumor), which have been subcutaneously transplanted into inbred Fischer rats, is inhibited by application of a single dose of ionizing radiation of 10 Gy or more (Cerniglia G J, Wilson D F, Pawlowski M, Vinogradov S, Biaglow J: Intravascular oxygen distribution in subcutaneous 9L tumors and radiation sensitivity. J Appl Physiol (1985) 82: 1939-45, 1997.). Moreover, according to other previous studies, it had been reported that the dose of ALA by a single intravenous injection is 100 to 500 mg/kg body weight in the photodynamic therapy (PDT) using rodents (Yamamoto J, Yamamoto S, Hirano T, Li S, Koide M, Kohno E, et al.: Monitoring of singlet oxygen is useful for predicting the photodynamic effects in the treatment for experimental glioma. Clin Cancer Res 12:7132-9, 2006; Abels C, Heil P, Dellian M, Kuhnle G E, Baumgartner R, Goetz A E: In vivo kinetics and spectra of 5-aminolaevulinic acid-induced fluorescence in an amelanotic melanoma of the hamster. Br J Cancer 70: 826-33, 1994; Abels C, Fritsch C, Bolsen K, Szeimies R M, Ruzicka T, Goerz G, et al.: Photodynamic therapy with 5-aminolaevulinic acid-induced porphyrins of an amelanotic melanoma in vivo. J Photochem Photobiol B 40: 76-83, 1997; Bozzini G, Colin P, Betrouni N, Maurage C A, Leroy X, Simonin S, et al.: Efficiency of 5-ALA mediated photodynamic therapy on hypoxic prostate cancer: a preclinical study on the Dunning R3327-AT2 rat tumor model. Photodiagnosis Photodyn Ther 10: 296-303, 2013.). Accordingly, in the present study, the present inventors have determined the maximum dose of ionizing radiation to be 10 Gy, and the maximum dose of ALA to be 500 mg/kg body weight. To date, the present inventors had demonstrated that the growth of tumor can be suppressed in vitro by combining application of multiple doses of ionizing radiation with ALA administration (Yamamoto J, Ogura S, Tanaka T, Kitagawa T, Nakano Y, Saito T, et al.: Radiosensitizing effect of 5-aminolevulinic acid-induced protoporphyrin IX in glioma cells in vitro. Oncol Rep 27: 1748-52, 2012.). Multiple intravenous injections into the caudal vein of rats had been technically difficult, but according to the experience of the present inventors, at maximum 5 times of injections had been possible. Accordingly, the present inventors have determined an optimal ionizing irradiation schedule, in which ionizing irradiation at a dose of 2 Gy/day and ALA multiple-dose administration at a dose of 100 mg/kg body weight/day were carried out for 5 consecutive days. After the subcutaneous tumor had grown to a size of 6 to 8 mm in diameter, the rats were randomly divided into four groups, and were then treated as follows:

control group (without treatments) (n=5);
ALA multiple-dose administration group (n=5);
ionizing radiation multiple-dose application group (RT) (n=7); and
ALA multiple-dose administration+ionizing radiation multiple-dose application group. (5-ALA+RT) (n=7).

In a group to which only ALA was administered, only ALA (100 mg/kg) was administered to rats via caudal vein for 5 consecutive days. In a multiple-dose application group involving only ionizing radiation, rats were anesthetized and were then irradiated with an ionizing radiation of 2 Gy/day for 5 consecutive days (a total of 10 Gy). In an ALA multiple-dose administration+ionizing radiation multiple-dose application group, for 5 consecutive days, rats were intravenously administered with ALA (100 mg/kg), then 3 hours later, the rats were anesthetized, and an ionizing radiation of 2 Gy/day was applied to the subcutaneous tumor thereof in a dark place (a total of 10 Gy). Ionizing radiation was applied at a rate of 0.65 Gy/min, using an X-ray irradiation apparatus (MBR-1520R; HITACHI Engineering & Service Co., Ltd., Japan). At this time, the body of each rat, other than the tumor site, was completely covered with an X-ray protective sheet, so that the excessive exposure of the remaining body portion to the ionizing radiation was avoided. In order to avoid photochemical effects, all of the rats were bred in a dark place for 12 hours after ALA administration. Thereafter, the direct exposure of the rats to room light was avoided. After completion of the treatment, the growth of the tumor was evaluated every other day until the 16th day. The tumor volume was calculated according to the formula: V=a²b/2 (wherein V indicates volume, and a and b indicate minor axis and major axis, respectively) (Niclou S P, Danzeisen C, Eikesdal H P, Wiig H, Brons N H, Poli A M, et al.: A novel eGFP-expressing immunodeficient mouse model to study tumor-host interactions. Faseb J 22: 3120-8, 2008.). On the 16th day after completion of the treatment, the rats were sacrificed under deep anesthesia. All of the tumor samples including the portion covering the dorsal skin were promptly excised, and were then immobilized in 20% formaldehyde/PBS for the subsequent pathologic examination.

### [Pathologic Examination]

After completion of the immobilization, all of the tumor samples were cut at the center of tumor in the longitudinal direction. The obtained fragments were each stained with hematoxylin and eosin (HE), and were also stained with Iba1 or CD68 for detection of macrophages. Briefly, the sample was bathed in water for pre-treatment (40 minutes, 95° C.), and the deparaffinized portion was then washed with KN Buffer (KN-09002; Pathology Institute Corp, Toyama, Japan). Subsequently, the fragment was incubated with goat polyclonal anti-Iba1 (1:3000, ab107159, Abcam) or anti-CD68 antibody (1:100, MCA-341R, AbD) for 30 minutes, and were further washed with KN Buffer. Subsequently, regarding Iba1, the fragment was incubated with Simple Stain™ MAX-PO (G) (H1301; Nichirei Bioscience Inc. Japan) for 30 minutes. Regarding CD68, the fragment was incubated with Envision+Dual Link HRP-label/polymer reagent (Dako: K4061) for 30 minutes. The resultant was finally washed with KN Buffer, and was then treated with diaminobenzidine (DAB) for 10 minutes for color development. The thus obtained fragment was subjected to counter staining with hematoxylin.

Moreover, the procedures of the previous study (Prall F, Maletzki C, Linnebacher M: Microdensitometry of osteopontin as an immunohistochemical prognostic biomarker in colorectal carcinoma tissue microarrays: potential and limitations of the method in ‘biomarker pathology’. Histopathology 61: 823-32, 2012.) were partially modified, and using public domain software, Image J (Wayne Rasband, National Institute of Mental Health, Bethesda, Md.), microdensitometry was carried out regarding immunohistochemical quantitative evaluation of Iba1 on a digital photomicrograph. In short, all of the Iba1-stained tumor samples were scanned using a color scanner, and were then photographed. In order to quantify the color signals of immunohistochemical DAB of Iba1, the image data of all samples were transferred into Image J, and were then converted to 8-bit grayscale images. Subsequently, regarding all of the tumor fragments, the entire section of the subcutaneous tumor in each tumor sample was described as a region of interest (ROI), using a freehand tool, and the mean gray value (MGV) in the ROI was obtained in the form of a histogram. The present inventors have defined the mean gray value (MGV) of all of the tumor samples in a control group (n=5) as a representative value, and the relative intensities of MGVs in other groups to the aforementioned representative value were then calculated.

### [Statistical Analysis]

Data were shown in the form of a mean value±SE. The mean volume of tumors was analyzed using Unpaired t-test, and the relative intensity of the MGV of Iba1 was calculated using Fischer exact probability test. Statistical significance was defined as p<0.05.

### Results

### [Accumulation of ALA-Induced PpIX in Tumor, in Rat Subcutaneous Tumor Models]

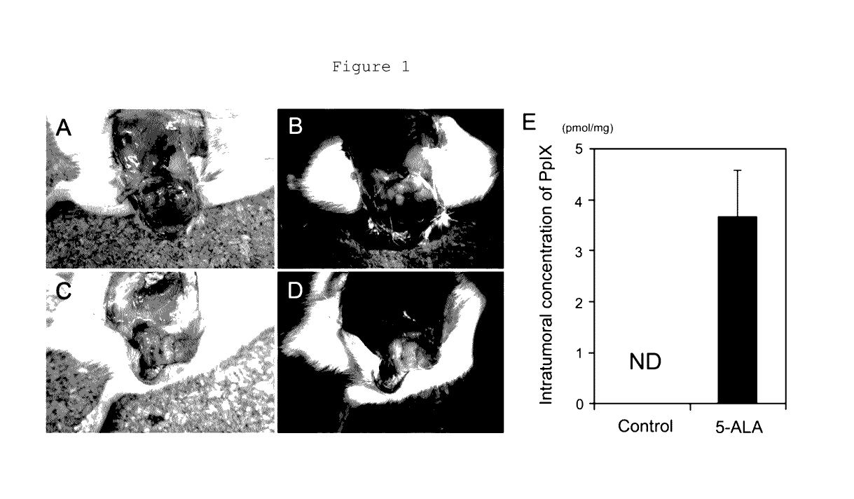

In the rat subcutaneous tumor models produced by the present inventors, the subcutaneous tumor 3 hours after the intravenous administration of ALA emitted strong fluorescence (FIG. 1D), in comparison to a control tumor to which ALA had not been administered (FIG. 1B).

Moreover, in an HPLC analysis, the amount of PpIX in tumor, which was induced by ALA 3 hours after the intravenous administration of the ALA, was 3.66±0.91 pmol/mg protein, and thus, it was shown that the amount was significantly larger than that of a control group (p<0.01), and high accumulation of ALA in the tumor was observed (FIG. 1E). On the other hand, such ALA-induced PpIX could not be detected in the tumor of the control group, to which ALA had not been administered (less than 0.1 pmol/mg protein).

### [Radiosensitizing Effects of ALA In Vivo, Upon Application of Multiple Doses of Ionizing Radiation]

Subcutaneously transplanted 9L gliosarcoma cells have grown at an almost exponential rate (FIG. 2, Table 1).

TABLE 1
Effect of 5-ALA and multi-dose ionizing irradiation on tumor growth

| Group | Day 0 | Day 8 | Day 10 |
|---|---|---|---|
| Control (n = 5) | 142.3 ± 13.8 | 1191.5 ± 128.8 | 1793.2 ± 199.2 |
| ALA (n = 5) | 143.9 ± 17.4 | 865.4 ± 107.2 | 1403 ± 224.3 |
| RT (n = 7) | 149.9 ± 14.9 | 683.3 ± 99.4 | 1059 ± 101.7 |
| ALA + RT (n = 7) | 146 ± 12.9 | 385.3 ± 59.7 | 495.6 ± 91.1 |
| p value* | 0.8457 | 0.0246 | 0.0014 |
| Group | Day 12 | Day 14 | Day 16 |
| Control (n = 5) | 2296.8 ± 233.3 | 2799 ± 281.9 | 3747.1 ± 254.1 |
| ALA (n = 5) | 1842.6 ± 218.8 | 2276.3 ± 192.8 | 2869.4 ± 267.9 |
| RT (n = 7) | 1485.8 ± 133.4 | 1736.9 ± 162.9 | 2035.2 ± 245.9 |
| ALA + RT (n = 7) | 867.6 ± 119.7 | 819 ± 135.5 | 863.1 ± 147.4 |
| p value* | 0.0007 | 0.001 | 0.0015 |

Data: mean tumor volume (mm³) ± SE
Blackets indicate number of sample.
*RT vs ALA + RT (unpaired t-test)

On the initial day of the treatment of tumor (Day 0), there was no difference in terms of tumor size between an ionizing radiation multiple-dose application group and an ALA multiple-dose administration+ionizing radiation multiple-dose application group (p=0.8457, Table 1). However, on the 10th day after the treatment, when compared with the ionizing radiation multiple-dose application group, tumor growth was significantly inhibited in the ALA multiple-dose administration+ionizing radiation multiple-dose application group (10th day onward, p<0.01) (FIG. 2, Table 1). On the 16th day after the treatment, when compared with the ionizing radiation multiple-dose application group, the mean tumor growth inhibitory rate was 42.5% in the ALA multiple-dose administration+ionizing radiation multiple-dose application group (FIG. 2A, Table 1). Interestingly, the growth of the tumor was inhibited even in the ALA multiple-dose administration group, in comparison to the control group (16th day, p=0.0448).

[Pathological Evaluation of Subcutaneously Transplanted 9L Gliosarcoma after Application of Multiple Doses of Ionizing Radiation]

With regard to tumor samples, coagulation necrosis was observed in subcutaneous tumor, although there were differences among the groups (FIG. 3A to FIG. 3D). Interestingly, also regarding Iba1-positive macrophages, there were differences among the groups (FIG. 3E to FIG. 3L). In the control group, extremely low amounts of Iba1-positive macrophages were only accumulated in subcutaneous tumors (FIG. 3E, FIG. 3I). In the ALA multiple-dose administration group, Iba1-positive macrophages were mainly gathered to the surface of the subcutaneous tumor, and some macrophages penetrated into the tumor (FIG. 3F, FIG. 3J). Likewise, many Iba1-positive macrophages were gathered to the surface and inside of the subcutaneous tumor after completion of the ionizing irradiation (FIG. 3G, FIG. 3K), and in particular, such gathering was significantly observed, after application of ionizing radiation, which was combined with ALA administration (FIG. 3H, FIG. 3L).

As a result of the microdensitometry analysis, it was revealed that the MGV in the ALA multiple-dose administration+ionizing radiation multiple-dose application group was significantly higher than the MGV in other groups, and that a larger number of Iba1-positive macrophages were gathered to subcutaneous tumor cells (p<0.05 in comparison to the ionizing radiation multiple-dose application group; p<0.01 in comparison to the ALA multiple-dose administration group) (FIG. 4A).

In addition, in the distribution analysis of Iba1-positive macrophages inside the tumor, it was observed that the Iba1-positive macrophages were not gathered to an area of coagulation necrosis, but were mainly gathered to a boundary zone between an area of coagulation necrosis and surviving tumor cells (FIG. 4B). Many Iba1-positive macrophages exhibited the characteristics of phagocytes and phagocytosis process (the arrows shown in FIG. 4B).

Moreover, even in the results obtained by performing immunostaining with an anti-CD68 antibody, the same tendency as that in the above described results was observed (FIG. 5).

### Consideration

Using glioma experimental models, the present inventors have demonstrated for the first time that ALA-induced PpIX has radiosensitizing effect in vivo.

A basic mechanism of the radiosensitizing effect of a porphyrin compound has not yet been elucidated. To date, using a confocal laser scanning microscope, the present inventors had demonstrated in vitro that ALA-induced PpIX in cells plays an important role for production of reactive oxygen species (ROS) and radiosensitization (Yamamoto J, Ogura S, Tanaka T, Kitagawa T, Nakano Y, Saito T, et al.: Radiosensitizing effect of 5-aminolevulinic acid-induced protoporphyrin IX in glioma cells in vitro. Oncol Rep 27: 1748-52, 2012.). This radiosensitizing effect can depend on the intracellular concentration of a porphyrin compound. However, it had been known that the intracellular concentration of a porphyrin compound, such as a hematoporphyrin derivative or photofrin, is low after completion of the ALA administration (Yamamoto J, Yamamoto S, Hirano T, Li S, Koide M, Kohno E, et al.: Monitoring of singlet oxygen is useful for predicting the photodynamic effects in the treatment for experimental glioma. Clin Cancer Res 12: 7132-9, 2006.). Accordingly, it has been assumed that, either in vitro or in vivo, a combination of single ionizing irradiation with a high concentration of hematoporphyrin derivative or photofrin in cells, would provide a stronger radiosensitizing effect, than a combination of single ionizing irradiation with ALA-induced PpIX (Luksiene Z, Juzenas P, Moan J: Radiosensitization of tumours by porphyrins. Cancer Lett 235:40-7, 2006; Berg K, Luksiene Z, Moan J, Ma L: Combined treatment of ionizing radiation and photosensitization by 5-aminolevulinic acid-induced protoporphyrin IX. Radiat Res 142:340-6, 1995; Schaffer M, Schaffer P M, Jori G, Corti L, Sotti G, Hofstetter A, et al.: Radiation therapy combined with photofrin or 5-ALA: effect on Lewis sarcoma tumor lines implanted in mice. Preliminary results. Tumori 88: 407-10, 2002; 24; Kostron H, Swartz M R, Miller D C, Martuza R L: The interaction of hematoporphyrin derivative, light, and ionizing radiation in a rat glioma model. Cancer 57: 964-70, 1986.). In several cell lines, ALA-induced PpIX would have provided only a low radiosensitizing effect, in the case of single ionizing irradiation (Yamamoto J, Ogura S, Tanaka T, Kitagawa T, Nakano Y, Saito T, et al.: Radiosensitizing effect of 5-aminolevulinic acid-induced protoporphyrin IX in glioma cells in vitro. Oncol Rep 27: 1748-52, 2012; Ito E, Yue S, Moriyama E H, Hui A B, Kim I, Shi W, et al.: Uroporphyrinogen decarboxylase is a radiosensitizing target for head and neck cancer. Sci Transl Med 3: 67ra7, 2011.). Moreover, another study had pointed out that application of ionizing radiation does not inhibit, but rather increases, in vitro, the synthesis of ALA-induced PpIX in human colon adenocarcinoma cells (Berg K, Luksiene Z, Moan J, Ma L: Combined treatment of ionizing radiation and photosensitization by 5-aminolevulinic acid-induced protoporphyrin IX. Radiat Res 142: 340-6, 1995.).

The present inventors have demonstrated that ALA-induced PpIX significantly increases the sensitivity of tumor to application of multiple doses of ionizing radiation. Therefore, by repeatedly applying ionizing radiation in combination with ALA administration, the radiosensitizing effect of ALA-induced PpIX is increased, so that the growth of tumor can be strongly inhibited.

Subsequently, the present inventors have conducted immunohistostaining using Iba1 to detect macrophages, and have examined the immune response of macrophages to ALA-induced PpIX, when ALA administration was combined with application of multiple doses of ionizing radiation. When ionizing radiation was applied several times, ALA-induced PpIX induced strong coagulation of Iba1-positive macrophages on the surface and inside of subcutaneous tumor. In general, the expression of Iba1 is typically up-regulated in activated macrophages/microglia, and shows a unique form having an ameba-like shape and short projection (David S, Kroner A: Repertoire of microglial and macrophage responses after spinal cord injury. Nat Rev Neurosci 12: 388-99, 2011; Lynch M A: The multifaceted profile of activated microglia. Mol Neurobiol 40: 139-56, 2009.). Macrophages can be broadly classified into the following two groups:

(1) Classically activated macrophages (M1): The classically activated macrophages are typically associated with coordinated response to immunogenic antigens, mainly mediated by production of proinflammatory mediators such as IL-1B, IL-12 and TNF-α, and generally improve an ability to englobe pathogenic substances (MacMicking J, Xie Q W, Nathan C: Nitric oxide and macrophage function. Annu Rev Immunol 15: 323-50, 1997; Boehm U, Klamp T, Groot M, Howard J C: Cellular responses to interferon-gamma. Annu Rev Immunol 15: 749-95, 1997.); and
(2) Alternatively activated macrophages (M2): It is considered that the alternatively activated macrophages do not secrete proinflammatory mediators such as IL-1B and TNF-α (Hussain S F, Yang D, Suki D, Grimm E, Heimberger A B: Innate immune functions of microglia isolated from human glioma patients. J Transl Med 4: 15, 2006.), have influence on immunomodulation, mainly mediated by secretion of strong immunosuppressive cytokines such as IL-10 and TGF-β, and decrease phagocytic ability (Li W, Graeber M B: The molecular profile of microglia under the influence of glioma. Neuro Oncol 14: 958-78, 2012; Filipazzi P, Huber V, Rivoltini L: Phenotype, function and clinical implications of myeloid-derived suppressor cells in cancer patients. Cancer Immunol Immunother 61: 255-63, 2012.).

In the present experiment, by applying ionizing radiation several times, in combination with ALA administration, many Iba1-positive macrophages were gathered to the surface and inside of subcutaneous tumor. In particular, Iba1-positive macrophages having the characteristics of phagocytes were mainly gathered to a boundary zone between an area of coagulation necrosis and surviving tumor cells. In contrast, a coagulation-necrotic change was observed in the subcutaneous tumor in each group, but almost no Iba1-positive macrophages were gathered to the surface of the subcutaneous tumor in the control group. These findings suggest that Iba1-positive macrophages be gathered not only for the purpose of treating coagulation-necrotic tissues, but be induced for antitumor purpose.

It had been reported that tumor-infiltrating mononuclear cells are increased in Lewis lung carcinoma by photodynamic therapy using ALA, and that intraperitoneal macrophages are activated in Lewis lung carcinoma-bearing mice (Skivka L M, Gorobets O B, Kutsenok V V, Lozinsky M O, Borisevich A N, Fedorchuk A G, et al.: 5-aminolevulinic acid mediated photodynamic therapy of Lewis lung carcinoma: a role of tumor infiltration with different cells of immune system. Exp Oncol 26: 312-5, 2004.). The present inventors have discovered that the growth of tumor growth is also inhibited by repetitive administration of ALA alone (FIG. 2B). The possibility that the aforementioned findings would have been obtained by photodynamic action cannot be completely eliminated. However, the exposure to room light was limited and indirect. Thus, the present inventors have found that ALA itself can induce or enhance the anti-tumor immune response of a host.

Patients having malignant brain tumor are frequently subjected to multifractionated radiation therapy, after completion of the surgical resection of tumor. Since various radiation modalities, such as stereotactic radiotherapy (SRT), stereotaxic radiosurgery (SRS), and intensity modulated radiotherapy, can precisely regulate the intensity of ionizing irradiation, they avoid or reduce the exposure of normal tissues, so that side effects caused by the therapy can be restricted. Accordingly, it becomes possible to precisely apply a high dose of radiation, for example, to a small region of intracranial lesion, according to SRT, SRS, etc. (Starke R M, Williams B J, Hiles C, Nguyen J H, Elsharkawy M Y, Sheehan J P: Gamma knife surgery for skull base meningiomas. J Neurosurg 116: 588-97, 2012; Torres R C, Frighetto L, De Salles A A, Goss B, Medin P, Solberg T, et al.: Radiosurgery and stereotactic radiotherapy for intracranial meningiomas. Neurosurg Focus 14: e5, 2003.). ALA has high affinity, for example, for a malignant brain tumor, and has many advantages such as low phototoxicity to skin, in comparison to other photosensitizers. The present inventors have found that application of multiple doses of ionizing radiation, in which ALA-induced PpIX is used, would become a novel therapeutic approach for brain tumors such as malignant glioma, and could be applied in clinical sites.

### CONCLUSION

Using glioma experimental models, the present inventors have demonstrated for the first time the radiosensitizing effect of ALA in vivo. By combining ALA administration with application of multiple doses of ionizing radiation, an anti-tumor immune response is induced or enhanced in the glioma experimental models, so that strong inhibition of tumor growth can be induced.

### Example 2

### Increase in Production of Delayed Reactive Oxygen Species (ROS) after Completion of Ionizing Irradiation, by ALA Administration

In the above described Example 1, the tumor immunity-inducing effect obtained by ALA administration was sufficiently demonstrated. However, just additionally, the present inventors have also examined the effect of ALA administration to increase production of delayed reactive oxygen species (ROS) after completion of ionizing irradiation.

### Materials and Methods

### [Chemical Substances]

5-ALA was purchased from Cosmobio (K.K., Tokyo, Japan), and for the subsequent in vitro studies, the purchased 5-ALA was then dissolved in a fresh culture solution to a final concentration of 1 mM. 2′,7′-Dichlorofluorescein diacetate (DCFD) was purchased from Sigma-Aldrich (K.K., Tokyo, Japan). DCFD was dissolved in a fresh culture solution or phosphate buffered saline (PBS)/fetal bovine serum (FBS) to a final concentration of 10 μM. Other substances were of the available highest grades.

### [Handling of Culture and Cells]

Two types of rat glioma cell lines (9L and C6) and human glioma cell lines (U251 and T98G) were used. Before use, 9L and T98G were cultured in RPMI-1640 at 37° C. for several days, whereas C6 and U251 were cultured in 10% FBS-added Dulbecco's modified Eagle's medium (DMEM) at 37° C. for several days. These cell lines were maintained at 37° C. in 5% CO2 in a humidified incubator. The cells were subcultured at an exponential growth phase thereof, using 0.05% trypsin solution containing 0.5 mM ethylenediamine tetraacetate. 70% confluent cells were used in the subsequent experiments. 5-ALA was dissolved in RPMI-1640 (9L and T98G), or in 10% FBS-added DMEM (C6 and U251), to a final concentration of 1 mM.

### [Evaluation of Intracellular Level of 5-ALA-Induced PpIX in Glioma Cells by Flow Cytometric Analysis]

The cells were seeded on a 100-mm culture dish, and were then cultured. The cells were incubated in a complete medium containing 1 mM 5-ALA for 4 hours, and were then washed with PBS. The resulting cells were peeled from the bottom layer by a trypsin treatment, and were then recovered by centrifugation (400×g, 3 min, and 4° C.). Immediately after the recovery, the cells were re-suspended in cold PBS/FBS, and were then analyzed using a flow cytometer (EC800; Sony Biotechnology, Tokyo, Japan). Generally, 30,000 cells contained in each sample were evaluated. Using 488-nm argon ion laser, fluorescence was excited from intracellular PpIX, and the fluorescence was then detected using a 640/30 nm band-pass filter. In order to prevent photoactivation of 5-ALA-induced PpIX, all procedures were carried out in a dark room. The flow cytometric data were analyzed using FlowJo (Tree Star Inc., Ashland, Oreg., USA). The mean fluorescence intensity (MFI) of PpIX fluorescence in the 5-ALA-treated cells, with respect to that of 5-ALA-not-treated cells, was calculated for each cell line.

[Evaluation of Cellular ROS Levels after Ionizing Irradiation to Glioma Cells]

Intracellular ROS production was evaluated using the oxidant-sensitive fluorescence probe DCFD and flow cytometry (Yamamori T, Yasui H, Yamazumi M, et al: Ionizing radiation induces mitochondrial reactive oxygen species production accompanied by upregulation of mitochondrial electron transport chain function and mitochondrial content under control of the cell cycle checkpoint. Free Radic Biol Med 53: 260-270, 2012.). The cells were seeded on a 100-mm culture dish, and using an X-ray irradiation apparatus (MBR-1520R; Hitachi, Tokyo, Japan) (0.67 Gy/min), the cells were then irradiated with a radiation of 10 Gy in a dark room. During the ionizing irradiation, the culture dish was maintained at a room temperature in a dark container. In the case of a 5-ALA and ionizing irradiation group (RT,12 h+ALA), the cells were treated with 1 mM 5-ALA for 4 hours, then, immediately after the treatment, the cells were irradiated with ionizing radiation, and were then incubated together with 10 μM DCFD at 37° C. for 15 minutes, followed by washing with PBS twice. Thereafter, as described above, the cells were analyzed using a flow cytometer (excitation: 488 nm; radiation: 525/50 nm band-pass filter). Control cells were not exposed to ionizing radiation or 5-ALA. In a 5-ALA group, the cells were treated with 1 mM 5-ALA for 4 hours, but were not exposed to ionizing radiation. The control cells and the cells of the 5-ALA group were treated with DCFD in the same manner as described above. In order to prevent the photoactivation of 5-ALA-induced PpIX and DCFD, all procedures were carried out in a dark room. The flow cytometric data were analyzed using FlowJo (Tree Star Inc., Ashland, Oreg., USA). The MFI of the DCF fluorescence in the cells, with respect to that in control cells, was calculated for each cell line.

[Detection of Intracellular Localization of ROS, 12 Hours after Application of Ionizing Radiation to Glioma Cells In Vitro]

Intracellular ROS production was detected by our method using DCFD and a confocal laser scanning microscope (LMS5 Pascal; Carl Zeiss, Jena, Germany) (Yamamoto J, Ogura S, Tanaka T, et al: Radiosensitizing effect of 5-aminolevulinic acid-induced protoporphyrin IX in glioma cells in vitro. Oncol Rep 27: 1748-1752, 2012.). Briefly, the cells were seeded in a fresh medium containing 1 mM 5-ALA in a 35-mm glass-bottom culture dish (Asahi Techno Glass, Tokyo, Japan), and were then incubated at 37° C. for 4 hours in a dark room. Thereafter, the cells were washed with PBS, and were then exposed to an ionizing radiation of 10 Gy. Twelve hours after the ionizing irradiation, the cells were washed with PBS twice, and were then incubated together with 10 μM DCFD for 15 minutes. Thereafter, the cells were washed with PBS twice, and immediately after the washing, the resulting cells were observed. DCFD fluorescence (excitation: 488 nm; radiation: 505-530 nm band-pass filter) was imaged on a confocal laser scanning microscope. All procedures were carried out in a dark room.

### [A Change in Delayed Cellular ROS Production by Performing 5-ALA Treatment on Glioma Cells at Different Time]

The cells were seeded on a 100-mm culture dish, and were then exposed to an ionizing radiation of 10 Gy. The incubation time (4 h) of 1 mM 5-ALA was the same among individual groups, and three types of 5-ALA treatment times were initiated: i) 5-ALA treatment immediately before ionizing irradiation [RT+ALA (pre) group]; ii) 5-ALA treatment immediately after ionizing irradiation [RT+ALA (4 h) group]; and iii) 5-ALA treatment 8 hours after ionizing irradiation [RT+ALA (12 h) group]. In each group, twelve hours after the ionizing irradiation, the cells were incubated together with 10 μM DCFD at 37° C. for 15 minutes, and were then washed with PBS twice. Immediately after the washing, as described above, DCF fluorescence was analyzed using a flow cytometer. Control cells were not treated with ionizing radiation or 5-ALA. In a group involving only ionizing irradiation (RT), the cells were exposed to ionizing radiation, without performing 5-ALA treatments, and were then treated with DCFD twelve hours after the exposure to the ionizing radiation in the same manner as described above. All procedures were carried out in a dark room. The flow cytometric data were analyzed using FlowJo software. The MFI of the DCF fluorescence in the cells, with respect to that in control cells, was calculated for each cell line.

### [Statistical Analysis]

The data were shown in the form of a mean value±SE, and were analyzed according to a Fischer's protected least significant difference test. p<0.05 was shown as a statistically significant result.

### Results

[Change Over Time in 5-ALA-Induced PpIX Production, after Ionizing Irradiation to Glioma Cells]

We have first studied the intracellular accumulation of 5-ALA-induced PpIX in glioma cell lines according to a flow cytometric analysis. In each cell line, the MFI of PpIX fluorescence in 5-ALA-treated cells was clearly increased, in comparison to a control (not shown in the figure). The relative MFI values of PpIX fluorescence (average±SE) in 9L, U251, C6, and T98G cells were 21.5±0.12, 37.6±0.12, 20.2±0.56, and 15.3±0.89, respectively. Among these cell lines, we have selected two cell lines showing relatively high relative MFI values (i.e. 9L and U251), and then, have used them in the subsequent studies. Subsequently, we have confirmed the influence of ionizing irradiation on production of 5-ALA-induced PpIX in the 9L and U251 cell lines (FIG. 6). In the case of the 9L cells, the relative MFI values (average±SE) of PpIX fluorescence 4, 12, and 24 hours after ionizing irradiation were 15.2±0.15, 15.9±0.73, and 13.4±2.43, respectively. There were no significant differences among individual groups (FIG. 6A). Also, in the case of the U251 cells, the relative MFI values (average±SE) of PpIX fluorescence 4, 12, and 24 hours after ionizing irradiation were 41.6±0.20, 44.2±3.67, and 51.1±3.76, respectively. There were no significant differences among individual groups (FIG. 6B).

[Delayed Increase in Intracellular Level of ROS, after Performing 5-ALA Treatment, Simultaneously with Ionizing Irradiation, on Glioma Cells]

FIG. 7 shows changes over time in intracellular ROS production after ionizing irradiation performed on 9L and U251 cells. In order to evaluate a direct interaction of 5-ALA-induced PpIX with ionizing irradiation on ROS production, the cells were pre-treated with 5-ALA, immediately before ionizing irradiation (FIG. 7A). In the case of 9L cells, the relative MFI values (average±SE) of DCF fluorescence immediately after (RT 0 h) 5-ALA administration (without ionizing irradiation) and ionizing irradiation without 5-ALA were 1.19±0.07 and 1.04±0.07, respectively, and there were no significant differences among all groups (p=0.383) (FIG. 7B and FIG. 7C). Twelve hours after ionizing irradiation without 5-ALA (RT 12 h), the relative MFI (1.41±0.13) of DCF fluorescence in the 9L cells was significantly higher than that of the RT 0 h group (p=0.047). However, the DCF fluorescence (1.91±0.19) 12 hours after ionizing irradiation with 5-ALA was apparently higher than that of the RT 12 h group (p=0.009). In the case of U251 cells, the relative MFI values (average±SE) of DCF fluorescence in 5-ALA administration (without ionizing irradiation) and the RT 0 h group were 0.87±0.06 and 0.99±0.03, respectively, and there were no significant differences among all groups (p=0.262) (FIG. 7D). The relative MFI (1.27±0.08) of DCF fluorescence in the RT 12 h group was significantly higher than that of the RT 0 h group (p=0.011). Likewise, the DCF fluorescence (1.51±0.10) 12 hours after ionizing irradiation with 5-ALA was apparently higher than that of the RT 12 h group (p=0.031).

[Delayed ROS Production in Glioma Cells after Ionizing Irradiation]

Cellular ROS after application of ionizing radiation to 9L and U251 cells was visualized based on DCF fluorescence, according to our method (Yamamoto J, Ogura S, Tanaka T, et al: Radiosensitizing effect of 5-aminolevulinic acid-induced protoporphyrin IX in glioma cells in vitro. Oncol Rep 27: 1748-1752, 2012.), using the oxidant-sensitive probe DCFD (FIG. 8). Previously, we had confirmed that there is no interaction between 5-ALA-induced PpIX and DCF fluorescence on a confocal laser scanning microscope (Yamamoto J, Ogura S, Tanaka T, et al: Radiosensitizing effect of 5-aminolevulinic acid-induced protoporphyrin IX in glioma cells in vitro. Oncol Rep 27: 1748-1752, 2012.). Twelve hours after ionizing irradiation, the DCF fluorescence was observed in the nucleus and the cytoplasm, and there was found a certain extent of difference in terms of the intensity of DCF fluorescence between the cell lines (see FIG. 8A-C for 9L cells, and FIG. 8G-I for U251 cells). In contrast, the pre-treatment of the cells with 5-ALA before ionizing irradiation apparently increased DCF fluorescence in the cytoplasm of the two types of cell lines (FIG. 8D-F and FIG. 8J-L).

[Influence of Difference in Terms of Timing of 5-ALA Treatment on Delayed Cellular ROS Production, in Glioma Cells after Ionizing Irradiation]

In order to evaluate the influence of different timings of 5-ALA treatments on delayed cellular ROS production, we have adopted three 5-ALA treatment timings, and have evaluated ROS production 12 hours after ionizing irradiation under individual conditions (FIG. 9A). In 9L and U251 cells, the DCF fluorescence in a 5-ALA treatment immediately before ionizing irradiation (RT+ALA (pre) group) (which was 1.93±0.10 in the 9L cells, and 1.44±0.02 in the U251 cells) was significantly higher than that of cells exposed to ionizing irradiation without 5-ALA treatments (which was 1.44±0.03 in the 9L cells, and 1.30±0.04 in the U251 cells) (p=0.0009 and 0.0135, respectively), and these results were matched with the results of the previous experiments (FIG. 9). In the case of the 9L cells, the DCF fluorescence in the cells in a 5-ALA treatment immediately before ionizing irradiation (RT+ALA (pre) group) was significantly higher than that (1.57±0.05) in the cells in a 5-ALA treatment performed immediately after ionizing irradiation (RT+ALA (4 h) group), and that (1.58±0.11) in the cells in a 5-ALA treatment performed 8 hours after ionizing irradiation (RT+ALA (12 h) group) (p=0.0072 and 0.0078, respectively) (FIG. 9B). The DCF fluorescence of the RT+ALA (pre) group in the U251 cells tended to be slightly higher than that of the RT+ALA (4 h) group (1.43±0.02) and that of the RT+ALA (12 h) group (1.38±0.05), but these were not significant differences (p=0.7958 and 0.2275, respectively) (FIG. 9C).

The aforementioned results have surprisingly demonstrated that 5-ALA administration to glioma cells, before ionizing irradiation, would strongly induce delayed ROS production in the cytoplasm after the ionizing irradiation.

From Example 1 and Example 2, it was demonstrated that ALA administration induces long-term immunological effects, such as an increase in delayed reactive oxygen species production or induction of tumor cytotoxic M1 macrophages, in tumor cells after completion of ionizing irradiation, and thus, it can strongly inhibit the growth of tumor. That is to say, the present inventors have found novel intended use of ALA as a tumor immunity-inducing agent for the first time. Clinically, because of the tumor immunity-inducing effect of ALA, persistent therapeutic effects can be expected for a certain period of time (for example, 2 to 3 months) after completion of irradiation (therapy) in combination with ALA.

1-14. (canceled)

15. A method for inducing tumor immunity in a subject affected with cancer, comprising:
(A) administering to the subject one or more therapeutically effective doses of a compound represented by the following formula (I): 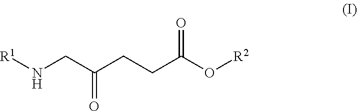
or a salt thereof,
wherein R¹ represents a hydrogen atom or an acyl group, and
wherein R² represents a hydrogen atom, a linear or branched alkyl group, a cycloalkyl group, an aryl group, or an aralkyl group; and
(B) optionally applying radiation to the subject one or more times.

16. The method according to claim 15, further comprising:
(C) administering an anticancer agent to the subject.

17. (canceled)

18. The method according to claim 15, further comprising administering an anticancer agent.

19. The method according to claim 15, further comprising administering an iron compound.

20. The method according to claim 19, wherein the iron compound comprises one or more compounds selected from the group consisting of ferric chloride, iron sesquioxide, iron sulfate, ferrous pyrophosphate, ferrous citrate, iron sodium citrate, ferrous sodium citrate, iron ammonium citrate, ferric pyrophosphate, iron lactate, ferrous gluconate, sodium iron diethylenetriamine pentaacetate, ammonium iron diethylenetriamine pentaacetate, sodium iron ethylenediamine tetraacetate, ammonium iron ethylenediamine tetraacetate, sodium iron dicarboxymethylglutamate, ammonium iron dicarboxymethylglutamate, ferrous fumarate, iron acetate, iron oxalate, ferrous succinate, sodium iron succinate citrate, heme iron, dextran iron, triethylenetetramine iron, lactoferrin iron, transferrin iron, sodium iron chlorophyllin, ferritin iron, saccharated iron oxide, and ferrous glycine sulphate.
